(19) United States
(12) Patent Application Publication
(10) Pub. No.: US 2023/0158135 A1
(43) Pub. Date: May 25, 2023

(54) IMMUNOGENIC COMPOSITION FORMING A VACCINE, AND A METHOD FOR ITS MANUFACTURE

(71) Applicant: ENGIMATA, INC, Pleasanton, CA (US)

(72) Inventors: Mitra Mosharraf, Danville, CA (US); Aryo Sorayya, Danville, CA (US); Rajiv Nayar, Danville, CA (US)

(73) Assignee: ENGIMATA, INC, PLEASANTON, CA (US)

(21) Appl. No.: 18/100,842

(22) Filed: Jan. 24, 2023

Related U.S. Application Data
(63) Continuation of application No. 17/684,075, filed on Mar. 1, 2022.
Application No. 17/684,075 is a continuation-in-part of application No. 17/204,511, filed on Mar. 17, 2021, now Pat. No. 11,559,577.
Application No. 17/204,511 is a continuation-in-part of application No. 16/925,438, filed on Jul. 10, 2020, now Pat. No. 11,278,617.
(60) Provisional application No. 63/003,254, filed on Mar. 31, 2020.

(51) Int. Cl. A61K 39/12; A61K 47/54; A61K 47/69; A61K 39/39; C12N 7/00; A61K 9/51

(52) U.S. Cl. CPC A61K 39/12; A61K 47/543; A61K 47/6911; A61K 47/6929; A61K 39/39; C12N 7/00; A61K 9/5123; A61K 2039/575

(57) ABSTRACT

A method of manufacturing an immunogenic composition forming a vaccine is disclosed. The method includes providing an antigen, providing a dry lipid blend, hydrating the dry lipid blend with an antigen solution, wherein the hydration is configured to form a colloidal vaccine solution, and extruding the colloidal vaccine solution, wherein the extrusion is configured to form a vaccine particle.

### CROSS-REFERENCE TO RELATED APPLICATIONS

This application is a continuation of Ser. No. 17/684,075, filed on Mar. 1, 2022, and entitled “IMMUNOGENIC COMPOSITION FORMING A VACCINE, AND A METHOD FOR ITS MANUFACTURE,” which is a continuation-in-part of U.S. Nonprovisional patent application Ser. No. 17/204,511, filed on Mar. 17, 2021, and entitled “IMMUNOGENIC COMPOSITION FORMING A VACCINE, AND A METHOD FOR ITS MANUFACTURE,” which claims priority to U.S. Nonprovisional patent application Ser. No. 16/925,438, filed on Jul. 10, 2020 and entitled “IMMUNOGENIC COMPOSITION FORMING A VACCINE, AND A METHOD FOR ITS MANUFACTURE,” which claims the benefit of priority of U.S. Provisional Patent Application Ser. No. 63/003,254, filed on Mar. 31, 2020 and entitled “LIPOSOMAL VACCINE ADJUVANT FOR VIRUS SPIKE PROTEINS AND METHODS OF MAKING AND USING SAME.” The entirety of U.S. Nonprovisional patent application Ser. No. 17/684,075, 17/204,511, 16/925,438 and U.S. Provisional Patent Application Ser. No. 63/003,254 are incorporated herein by reference.

### FIELD OF THE INVENTION

The present invention generally relates to the field of vaccine compositions and methods of making and using the same. In particular, the present invention is directed to an immunogenic composition forming a vaccine, and a method for its manufacture.

### BACKGROUND

Coronaviruses are an emerging pandemic threat that humans rarely have innate immunity to. Infection typically results in mild respiratory symptoms but can be more serious in infants and older adults, especially those with underlying comorbidities. Respiratory infection is second only to malaria as a cause of infant mortality worldwide and accounts for substantial hospitalization burden in both age groups in developed countries. Moreover, some pathogens, such as newly emergent zoonotic viral strains, can pose a significant risk of mortality to the general population as well.

### SUMMARY OF THE DISCLOSURE

In an aspect, a method of manufacturing an immunogenic composition forming a vaccine is disclosed. The method includes providing an antigen, providing a dry lipid blend, hydrating the dry lipid blend with an antigen solution, wherein the hydration is configured to form a colloidal vaccine solution, and extruding the colloidal vaccine solution, wherein the extrusion is configured to form a vaccine particle. In another aspect, a method of manufacturing an immunogenic composition forming a vaccine is disclosed. The method includes providing an antigen, providing a dry lipid blend, forming a nanoparticle delivery system, further comprising hydrating the dry lipid blend with a buffer, extruding the hydrated lipid blend, and combining the nanoparticle delivery system with the antigen, wherein the combination is configured to form a vaccine particle. The method further includes lyophilizing the nanoparticle delivery system and reconstituting the nanoparticle delivery system.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIGS. 1A-B is a schematic diagram of an exemplary embodiment of an immunogenic composition;

FIG. 2 is a schematic diagram of an exemplary embodiment of an immunogenic composition;

FIG. 3 is a schematic diagram of an exemplary embodiment of an immunogenic composition;

FIG. 4 is a schematic diagram of an exemplary embodiment of an immunogenic composition;

FIG. 5 is an exemplary diagram of combining a dried antigen and a dried nanoparticle adjuvant;

FIG. 6A-B is an exemplary embodiment of an injection device to administer a vaccine;

FIG. 7 is an exemplary embodiment of an injection device to administer a vaccine;

FIG. 8 is an exemplary embodiment of an injection device to administer a vaccine;

FIG. 9 is an exemplary embodiment of an injection device attachment to administer a vaccine;

FIG. 10 is a flow diagram illustrating an exemplary embodiment of a method for manufacture of an immunogenic composition;

FIG. 11 is a flow diagram illustrating an exemplary embodiment of a method for manufacture of an immunogenic composition;

FIG. 12 is a flow diagram illustrating an exemplary embodiment of a method for manufacture of an immunogenic composition;

FIG. 13 is a bar graph illustrating experimental results describing relative immunogenicity;

FIG. 14 is a bar graph illustrating experimental results describing relative immunogenicity;

FIG. 15 is a bar graph illustrating experimental results describing relative immunogenicity;

FIG. 16 is a bar graph illustrating experimental results describing relative immunogenicity;

FIG. 17 is a bar graph illustrating experimental results describing stability over time; and

FIGS. 18 and 19 are histograms illustrating experimental results describing stability over time. The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

Embodiments disclosed herein present a novel vaccine designed against spike proteins from coronaviruses, such as SARS-CoV-2, using a lipid-based nanoparticle nucleic acid formulation. Formulation may include a liposomal formulation. A resulting vaccine may be scalable, flexible in its antigen presentation, and have the potential for stability outside the cold chain. In an embodiment, a vaccine may include a positively charged chemical vaccine additive for cell targeting and may include a liposomal vaccine delivery system with entrapped, embedded, and/or surface adsorbed nucleic acids encoding viral spike proteins and protein complexes of a variety of viruses belonging to the Coronaviridae family of viruses for efficient presentation of the viral spike proteins to the immune system. This presentation of the viral spike protein antigen may induce a strong immune response in vivo and lead to the generation of coronavirus-neutralizing antibodies and significant amelioration of infection to coronaviral infections.

Embodiments may include, as a non-limiting example, a liposomal or other lipid-based nanoparticle vaccine formulation that includes entrapped, embedded, and/or surface adsorbed nucleic acids, which may encode a variety of viral proteins, such as the surface exposed glycoproteins (spike proteins) of the SARS-CoV-2 virus, S1 and/or S2. These nucleic acids may encode forms of S1, S2, and/or combinations therein (such as a polycistronic form relating to the native genomic mRNA sequence, and/or a fused form where the separate proteins are encoded as a single polypeptide), which may adopt various oligomeric states, found on enveloped viruses such as coronaviruses. “Spike proteins” are glycoproteins responsible for binding to host cell surface receptors and subsequent viral entry and represent a preeminent source of potential antibody-recognizing antigens. These spike protein complexes are believed to elicit a protective adaptive immune response in generating neutralizing antibodies against the viral surface, resulting in antibody opsonization and prevention of viral-mediated entry into host cells via spike protein interactions with host cell receptors. A potential avenue to combat such viruses may thus be to create a vaccine against these spike proteins, and other similar glycoproteins, which have been extensively characterized for other human coronavirus such as SARS and MERS, as well as non-human animal coronaviruses such as PEDV, FPIV, and MHV. Presentation of these antigenic glycoproteins in a more physiologically relevant, lipid-associated presentation to the immune cells may be essential to eliciting an appropriate immune response.

The pandemic caused by the Severe Acute Respiratory Syndrome Coronavirus 2 (SARS-CoV-2), previously known as the 2019 novel coronavirus (2019-nCoV) is an example of such an enveloped virus that has a trimeric spike (S) protein at its viral surface. The trimeric S protein of SARS-CoV-2, consisting of an S1 protein and a S2 protein, is responsible for binding to the host cell surface receptor, angiotensin-converting enzyme 2 (ACE2), and trigger subsequent receptor-mediated viral entry into the host cell. Symptoms in infected patients include fever, coughing, malaise, night sweats, headache, and breathing difficulties that may ultimately be fatal, especially in elderly patients or those with underlying diseases. Additionally, there are human and non-human coronaviruses with significant health and/or economic impact or potential impact including, without limitation, SARS (Severe Acute Respiratory Syndrome), MERS (Middle Eastern Respiratory Syndrome), MHV (Mouse Hepatitis Virus), PEDV (Porcine Epidemic Diarrheal Virus), and FIPV (Feline Infectious Peritonitis Virus); the last three infect only non-human animals, but boast high mortality rates and rates of infection and attack economically and scientifically important species.

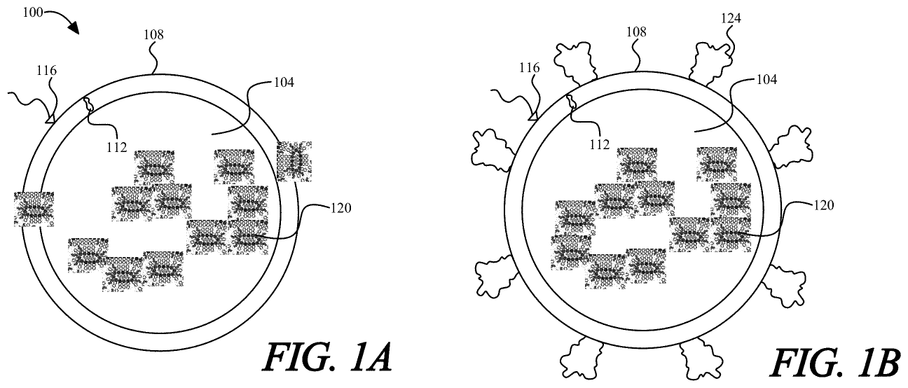

Referring now to FIG. 1A, an exemplary embodiment of an immunogenic composition 100 is illustrated. Immunogenic composition 100 includes a nanoparticle delivery system. A “delivery system,” as used in this disclosure, is an object or plurality of objects used to deliver an antigen, as defined below, to a-location within living tissue, a living organism such as a human, or the like; the intended location may include, for instance, one or more immune cells, one or more locations within the one or more immune cells, one or more cells that may act as a host for protein transcription, or any other location that may occur to a person skilled in the art upon reviewing the entirety of this disclosure. A delivery system may include, in a non-limiting example, an adjuvant. An “adjuvant,” as used in this disclosure, is a pharmacological and/or immunological agent that improves, or helps to stimulate, an immune response of a vaccine, antigen, or other immunologically active compound. Nanoparticle delivery system includes at least a nanoparticle 104. A “nanoparticle,” as used in this disclosure, is a particle of matter between 1 and 2000 nanometers in diameter. For instance, and without limitation, at least a nanoparticle 104 may be engineered to have an average size less than 500 nm in diameter. At least a nanoparticle 104 may be engineered to have a diameter between 50 nanometers and 2000 nanometers. At least a nanoparticle may have a diameter between 50 nanometers and 1000 nanometers. At least a nanoparticle may have an average diameter of approximately 200-300 nanometers. At least a nanoparticle may include a plurality of particles, a large majority of which are between 80 nanometers and 500 nanometers in diameter; a small number of outliers may be between 5 nanometers and 1200 nanometers. At least a nanoparticle 104 may include a plurality of nanoparticles, which may be suspended, without limitation, in an aqueous medium, lyophilized, and/or cryogenically preserved as described in further detail below. At least a nanoparticle 104 includes a lipid layer 108 exterior including a plurality of lipids, which may vary in physicochemical properties. Lipid layer 108 exterior may include, without limitation, a lipid monolayer, bilayer, and/or multi-lamellar construction and/or lipid corona about a non-liposome nanoparticle, which may include any nanoparticle as described above, or the like. At least a nanoparticle 104 may include, for instance, a liposome. A “liposome,” as used in this disclosure, is a vesicle enclosed by a lipid and/or phospholipid bilayer. Alternatively or additionally, at least a nanoparticle 104 may include a micelle, defined as a lipid monolayer enclosure, a micelle, an amphipol, a nanodisc, a styrene-maleic acid lipid particle (SMALP), and/or a nanostructure such as a piece of inorganic and/or organic material such as metal-based, metal oxide, carbon-based, immune-stimulating complex (ISCOM), protein cages, or other nanoparticles with a lipid material, or the like. Lipid and/or lipids making up lipid layer 108 and/or nanoparticle 104 construction may include, without limitation, phospholipids such as dipalmitoyl phosphatidylcholine (DPPC), dioleoyl phosphatidylcholine (DOPC), non-phospholipid lipids incorporating and/or combined with polyethylene glycols (PEGs), such as without limitation, PEGylated lipids, PEG-conjugated lipids, or the like, zwitterionic, neutral, cationic, and/or anionic phospholipids and non-phospholipids such as phosphatidylcholine, ceramides, phosphatidylethanolamine, saturated, monounsaturated and/or polyunsaturated fatty acid lipids, and the like. Lipids may be selected from the FDA GRAS list for approved excipients, for instance to guard against any biosafety issues. Lipid layer 108 includes cholesterol 112 and/or cholesterol 112 derivatives, including cholesterol with other functional groups added on; cholesterol derivatives may include, without limitation, cholesterol derivatives denoted as disterol-phospholipid Bis-Azo-PC, Chol-T, Chol-Q, or the like. Lipid layer 108 includes a primary alkyl amine 116, defined as a structure having an amine functional group and one or more carbon tails in an unbranched and/or branched carbon chains formation; primary alkyl amine 116 may include without limitation nonadecanamine, stearylamine, heptadecylamine, cetylamine, tripentyalmine, and/or isomers of the alkyl amines, or the like.

Continuing in reference to FIG. 1A, primary alkyl amine 116 includes a positively charged amino group head and at least a carbon tail. Non-limiting examples of primary alkyl amine 116 include stearylamine (SA), pentylamine (C₅H₁₃N), alkyl amines of any carbon length, as well as branched alkyl amines such as tripentyalmine, amylamines, or the like, mixtures of isomers of the above, and/or alkyl amines with varying degrees of poly- and mono-unsaturated carbon chains, such as alkene and/or alkyne substituted alkyl amines. Primary alkyl amine 116 may be positively charged. As a result, lipid layer 108 and/or at least a nanoparticle 104 may be positively charged; in an embodiment, positive charge of primary alkyl amine 116 may neutralize a net negative charge of at least a nanoparticle 104 and/or may cause overall charge of at least a nanoparticle 104 to become positive. In an embodiment, and without limitation, where at least a nanoparticle 104 is positively charged, at least a nanoparticle 104 may attract spike proteins having negative charges, improving entrapment and/or adsorption to lipid surface of spike protein. In a non-limiting example, a positive charge of combined nanoparticle and antigen may further have an effect of attraction to negatively charged cell membranes of immune and/or somatic cells, which may cause combined nanoparticle and antigen to contact and/or deliver into such cells the antigens; this may increase immunogenic effect of the resulting vaccine by improving cell-targeting. In some embodiments, spike proteins may alternatively or additionally complex bind to lipid layer; for in instance, spike protein may interact and change protein conformation to affect a complex bind, which may occur as a non-limiting example where a formulated vaccine is lyophilized and then reconstituted. In some embodiments, where antigen has a positive charge, alkyl amine and/or an additional compound having a negative charge, such as without limitation DPPG (dipalmitoyl, dioleoyl, disterylphosphatidylglycerol), alginate, and/or polyalginate, may be used to give lipid layer a net negative charge. Generally, where antigen has an electric charge with a first polarity, lipid layer exterior may have an electric charge with a second polarity, wherein the first polarity differs from the second polarity; i.e. where the first polarity is negative the second polarity may be positive, and vice-versa.

Still referring to FIG. 1A, as a non-limiting example, materials used in lipid layer 108 and/or liposome may include cholesterol 112 at approximately 20 mol %, saturated lipids DPPC in an amount of approximately 20-40 mol %, SA, positively charged, at approximately 15-45 mol %, and unsaturated lipid DOPC neutral, at approximately 15-25 mol %. In a non-limiting, illustrative embodiment, ratios of lipids may be in a range of DPPC:DOPC: cholesterol 112: alkyl amine molar ratio is 20-40:15-30:20:10-45. In an embodiment, differing molar ratios may be used to optimize various recombinant forms of spike proteins, and/or improve adsorption of coronavirus spike proteins from other species.

Further referring to FIG. 1A, immunogenic composition 100 includes an antigen incorporated in the at least a nanoparticle 104. An “antigen,” as used in this disclosure, is a viral molecule and/or molecular structure that may induce an antigen-specific antibody response and/or result in immune cell antigen receptor-binding that may be encoded in a nucleic acid sequence. Antigen, as used to herein, may refer to an antigenic protein and/or a nucleic acid encoding for an antigenic protein, a portion of an antigenic protein, a portion and/or entirety of a protein complex, or the like; more generally, nucleic acid may encode any protein, portion of protein, and/or chain of one or more amino acids. A “nucleic acid,” as used in this disclosure, is a biomolecule consisting of at least a nucleotide. Nucleic acid 120 may include DNA and/or RNA macromolecules, which may be present as single-stranded (ss), double stranded (ds), circular, linear, supercoiled, relaxed, nicked, or in any other form nucleic acids may adopt to be packed and/or arranged in immunogenic compositions. Nucleic acid 120 may include any type of nucleic acid such as antisense oligonucleotide, small interfering RNA (siRNA), mRNA, plasmid DNA (pDNA), and the like. Nucleic acid may elicit an immune response, without limitation, by being transcribed into one or more proteins, such as spike proteins or the like as described in this disclosure.

Continuing in reference to FIG. 1A, nucleic acid may encode an S2 protein. Alternatively or additionally, nucleic acid may encode an S1 protein. In non-limiting exemplary embodiments, nucleic acid 120 may include positive sense (+)RNA molecules which may be translated directly into a polypeptide once entered into a cell, such as mRNA. Such nucleic acid 120 may mimic coronavirus genomic RNA as positive sense (+)RNA, which may be directly translated into a polypeptide in the cellular cytosol after internalization. In this way, the mRNA molecule is translated into at least a copy of the viral protein and then subsequently degraded, after some time, in the cytosol of the cell. Thus, the antigen is a biomolecular precursor, which is used as an mRNA template for ribosomal translation into viral-mimicking peptides. The nucleic acid acts as a pharmacologically active synthetic drug which is converted into protein after internalization into a cell.

In an embodiment, combination of antigen such as nucleic acid with a delivery mechanism as described in this disclosure may obviate any need to use solvents such as ethanol in generating a composition. Combination of positively charged lipids with negatively charged nucleotides such as RNA, and/or reconstituting solution of one or other with the other one, may enable composition without use of solvents during mixture and/or reconstitution, for instance as described below.

Referring now to FIG. 1B, in an embodiment, antigen may also include a spike protein from a coronavirus, which may include any virus in the subfamily Orthocoronavirinae. A “spike protein,” as used in this description, is a protein and/or glycoprotein structure that projects from, lies on, and/or traverses a surface of a virus particle. A spike protein in a coronavirus may be referred to as an “S” protein, for instance S1 or S2. Spike protein 124 may include without limitation a trimeric protein complex or one or more subunits thereof, such as an S1 subunit, an S2 subunit, or the like, or homo- and/or hetero- oligomeric forms of these proteins. In an embodiment, and as described in further detail below, an S2 subunit may be embedded in a lipid bilayer of a virus particle, while a corresponding S1 subunit may bind to the S2 protein and project beyond the bilayer, extending away from the virus particle surface to engage host cells; this may enable a coronavirus to penetrate such cells by binding, for instance, the human ACE2 receptor, leading to internalization of the virus particle and/or a payload thereof, and ultimately infection. Spike protein 124, and/or any sub-unit thereof as described above may contain at least a post-translational modification (PTM) such as glycosylation, phosphorylation, acetylation, ubiquitination, isoprenoid attachment, or the like. Spike protein 124 may be recombinant, and/or may be harvested from partial and/or whole viral particles. For instance, and without limitation, spike protein may include NCP-CoV (2019-nCoV) spike protein (S1+S2 ECD) and/or SARS-CoV-2 (2019-n-Cov) Spike S1-His recombinant protein. Spike protein 124 may include a His tag; in such an example, a ‘His tag’ may be a poly-histidine amino acid fusion tag, as part of a recombinant spike protein, used for purification of the recombinant spike protein. Recombinant spike protein forms may contain purification tags, artifacts, or the like, including histidine tags, maltose-binding protein (MBP) tags, streptavidin-biotin tags, FLAG tags, and the like. Recombinant spike proteins and/or any viral glycoproteins used in nanoparticle formulations may originate from prokaryotic and/or eukaryotic recombinant expression systems, for instance and without limitation, mammalian cell expression, bacterial cells expression, yeast cell expression, and insect cell-baculoviral expression systems, and the like. Recombinant spike proteins and/or viral glycoproteins may be modified in DNA sequence to optimize recombinant expression and/or purification, but still result in faithfully recapitulated amino acid sequences resembling native viral proteins. Spike protein 124 may be HPLC-verified. Persons skilled in the art, after reviewing the disclosure in its entirety, will be aware of the various forms purified recombinant viral proteins may present.

With continued reference to FIG. 1B, a spike protein 124 or other antigen may include, without limitation, a glycoprotein. A glycoprotein is a surface-exposed viral structural protein that contains glycans—carbohydrate PTMs on the surface and/or within the protein. An S1 glycoprotein of a coronavirus may be, without limitation a homotrimer, a monomer, and/or a dimer. A process whereby glycans are chemically modified onto a surface of a glycoprotein is referred to as the process of “glycosylation,” and is a post-translational modification (PTM) defined as a chemical attachment to a protein after synthesis in the cell. Glycosylation may function to shield, or otherwise alter, antigenic sites on a virus for immune cell avoidance. Different glycosylation states may exist for glycoproteins such as SARS-CoV-2 glycoproteins, including without limitation other PTMs such as hydroxylation, methylation, lipidation, acetylation, disulfide bond formation, ubiquitination, SUMOylation, phosphorylation, proteolysis, and the like, as described above. Depending on the recombinant source, there may be final glycosylation states that differ in their modification pattern, amount, branching, and physicochemical properties, and potentially their immunogenicity; for instance, different forms of glycosylation may result from recombinant production of spike proteins in insect, mammalian, bacterial, and yeast cells or other organisms used for recombinant manufacture of the spike protein. In some embodiments, spike proteins used may evince varying truncated and/or mutated forms such as forms having various amino acid mutations.

Further referring to FIG. 1B, in alternative embodiments, antigen may include one or more surface proteins of other types of viruses, such as without limitation influenza virus or respiratory syncytial virus (RSV). Antigen may alternatively or additionally include surface proteins besides spike proteins, such as “M” proteins; in an embodiment, use of a mixture of S proteins and M proteins may modify and/or improve overall immunogenicity, stability, glycoprotein packing, or the like.

Continuing in reference to FIG. 1B, it is important to note that liposome-based immunogenic composition 100 for generating adaptive immune response in humans from, for instance SARS-CoV-2, may incorporate combinations of nucleic acid 120 payloads and liposome-incorporated spike protein 124. In this way, the formulation may provide spike proteins directly for antigen processing, as well as a template nucleic acid for generating additional antigens. This may represent a strategy for increasing 1) the immunological kinetics where there is a short burst of viral antigens present followed by a slower development of viral antigens as the mRNA is being translated. The immunological kinetics allow for Toll-Like Receptors (TLRs) and/or MHC Receptors to generate stable interactions with the viral proteins initially provided. Secondarily, additional viral peptides may be translated after the first “batch” of viral proteins is processed. And 2) potentially allow for multi-epitopes. Viral proteins translated from mRNA may have the benefit of multi-directionality where all surfaces are outwardly exposed for recognition. The polarity of the viral protein may not necessarily be maintained, where there is no lipid-embedded side and solvent-accessible side.

Continuing in reference to FIG. 1B, mRNA may comprise a first half-life in the cell, and the spike protein a second half-life, wherein the half-lives differ enough to provide temporal differences in immunology kinetics. The mRNA may thus be degraded soon after being translated, whereas the spike protein may be processed much quicker and prior to translation of mRNA. This way each antigen may be present only as long as necessary under normal cellular conditions to prepare viral proteins for display to immune cells. Nanoparticle 104 immunogenic compositions may have the benefit of encapsulating nucleic acid 120 antigens which encode for viral components. Nucleic acid 120 may include mRNA sequences for coronavirus spike protein 124 S1 and/or S1S2, as described herein. In such an instance, the nucleic acid 120 acts as an antigen precursor, which is translated into the de facto recombinant viral glycoprotein (surface antigen) which may then be recognized as the true antigen for which an adaptive immune response will be mounted.

Alternatively and/or additionally, and with continued reference to FIGS. 1A-B, nucleic acid 120 may be used in lieu of spike protein 124 for eliminating unnecessary antigenic load, potentially decreasing chances of allergic responses. For instance and without limitation, nucleic acid 120 may encode short immunogenic peptide fragments with the ability to elicit strong and targeted immune responses, avoiding the chances of allergenic reactions and/or secondary immunogenic effects. This way, the need for spike protein 124 may be circumvented and replaced with libraries of short peptides which are anticipated to generate strong immunogenic response without encoding for the fully functional spike protein 124.

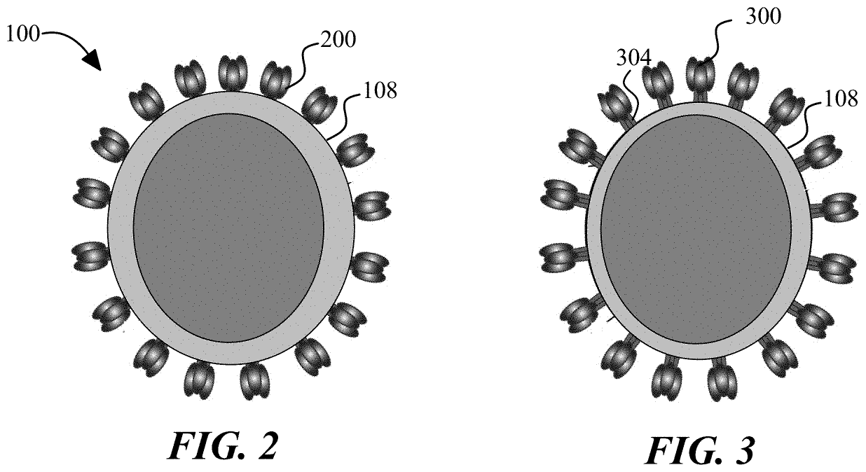

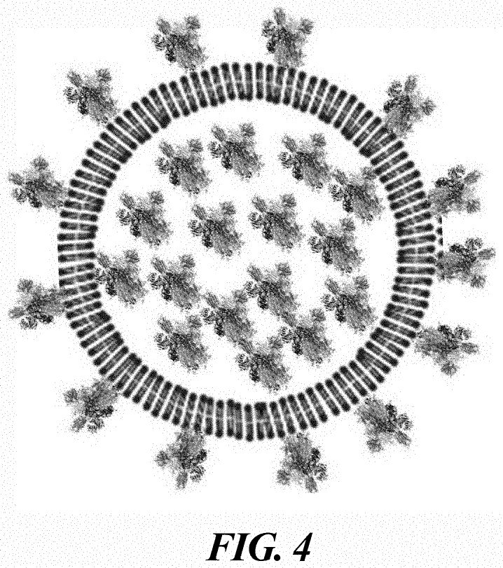

Referring now to FIG. 1B, antigen is incorporated in the at least a nanoparticle 104. “Incorporation,” as used herein, is any form of attachment, adsorption, and/or entrapment on or in a nanoparticle; for instance, and without limitation, antigen may be adsorbed to a surface of lipid layer 108. As a non-limiting example, and as shown in FIG. 2, spike protein may include an S1 protein 200 without an S2 in complex with it, which may be attached to and/or adsorbed to lipid layer. As a further non-limiting example, and as illustrated in FIG. 3, spike protein may include an S2 protein 300 embedded in lipid layer 108, adsorbed to lipid layer 108 and/or bilayer, and/or interacting with lipid layer 108 and/or bilayer, and an S1 protein 304 projecting from the lipid layer 108. As a further non-limiting example, and as shown in FIG. 4, where nanoparticle includes or is a liposome, spike protein 124 may be entrapped in an aqueous compartment of the liposome, and/or may be adsorbed to lipid layer as well. Incorporation may include entrapment between layers of a bilayer; for instance, where lipid layer 108 includes a bilayer and/or multi-lamellar construction, spike protein may be entrapped within the bilayer.

Referring now to FIG. 1A, incorporation of nucleic acid 120 in nanoparticle 104 may include attachment and/or adsorption of nucleic acid 120 onto the surface of the nanoparticle 104. For instance and without limitation, such a nanoparticle 104 may use positively charged surface treatment, for instance with primary alkyl amine 116, such as stearylamine. Incorporation of nucleic acid 120 in nanoparticle 104 may include entrapment of nucleic acid 120 within the aqueous core of the nanoparticle 104. In an embodiment, combination of negatively charged nucleic acids such as RNA with positively charged lipids, liposomes, and/or nanoparticles may facilitate combination, entrapment, and/or attachment of the nucleic acids to, with, complexed with, and/or in the positively charged lipids, liposomes, and/or nanoparticles. This may be accomplished, in exemplary embodiments, without use of ethanol or other volatile solvents. A resulting combination may be positively charged, further attracting and/or being attracted to a negatively charged surface and/or target location. Alternatively or additionally, a resulting combination may be negatively charged, further attracting and/or being attracted to a positively charged surface and/or target location.

Continuing in reference to FIGS. 1A and 1B, in an embodiment, entrapment may improve nanoparticle 104 stability by housing nucleic acid 120 within an aqueous core. In an embodiment, nanoparticle 104 may have an aqueous and/or hydrophilic core; alternatively, nanoparticle may have many nested layers of lipids, between which antigen may be entrapped and/or with hydrophilic elements of which antigen may be combined and/or complexed. Although, it is anticipated that there may be stability issues with nucleic acid adsorbed onto the surface of a nanoparticle, such stability challenges may be found in the form of nucleases (freely circulating endo- and exonucleases), presence of reactive oxygen species, among other endogenous and exogenous reactive species, potential for degradation and modification with factors present within the blood, tissues, and the like. Alternatively or additionally, nucleic acid 120 may traverse the lipid bilayer 108 and/or be embedded within lipid bilayer 108. For instance, as depicted in FIGS. 1A and 1B, nucleic acid embedded within a unilamellar lipid structure where the polarity of some lipids solvating the nucleic acid are flipped such that ionizable groups may be in contact with the nucleic acid, and carbon chains outwardly facing. Nucleic acid 120 may be in complex with any lipid as described herein, including for instance ionizable amino-lipids such as dilinoleylmethyl-4-dimethylaminobutyrate, DLin-MC3-DMA, and the like, “helper” lipids such as 1,2-distearoyl-sn-glycero-3-phosphocholine, DSPC, and the like, PEGs and/or PEGylated lipids such as 1,2-dimyristoyl-sn-glycerol, methoxypolyethylene glycol, PEG-DMG, among others, and/or cholesterol and/or cholesterol derivatives. Nanoparticle 104 for entrapment of nucleic acid 120 based immunogenic composition 100 may include nucleic acid particles solvated within a unilamellar lipid layer, among other lipid arrangements, within an aqueous core. Nanoparticle 100 may include viral glycoprotein and/or spike protein 124 antigen alone, nucleic acid 120 antigen alone, or combinations thereof. Nanoparticle 100 formulation may include nucleic acid 120 of multiple types of the same viral peptide. For instance and without limitation, nucleic acid 120 may include several mRNA transcripts for the pre-fusion glycoprotein, native glycoprotein, post-fusion form, individual small peptide sequences relating to various epitopes, among other forms. In further non-limiting illustrative examples, nucleic acid 120 may include mRNA corresponding to a plurality of different viral proteins, such that an immune response may be mounted against several different viral antigens. In either case, a more robust repertoire of epitopes for each antigen, or collection of antigens, may be used to cultivate a stronger, longer lasting adaptive immunological response. Nanoparticles 104 for nucleic acid 120 delivery may additionally differ in their lipid composition, surface properties, and size within the context of the physicochemical properties described herein. In an embodiment, combination of nanoparticles and antigens may be driven and/or enabled by ionic interaction due to opposite charges, for instance as described below, which may occur without limitation in hydrophilic portions of membrane or in core of a liposome, where nanoparticle includes a liposome. Antigen may complex with one or more parts of phospholipids, with one or more parts of lipids, and/or other elements of nanoparticle such as alkyl amine and/or stearylamine or other chemical components of nanoparticle.

With continued reference to FIGS. 1A and 1B, incorporation may be achieved by optimizing, or otherwise altering, the lipid composition, surface charge of the nanoparticle 104, and size of the nanoparticle 104, as well as other physicochemical properties. For example, an antigen such as an S1S2 spike protein of SARS-CoV-2 may be a negatively charged protein, for instance with acidic patches, that binds more efficiently to positively charged lipid surfaces and/or liposomes through favorable ionic interactions. Therefore, mixing a positively charged nanoparticle 104 such as a positively charged liposome with an S1S2 spike protein of SARS-CoV-2 may result in protein adsorption to the liposome and/or nanoparticle surface as well as some entrapment inside the liposome and/or nanoparticle. This particle-protein complex may subsequently interact with the immune cells and elicit a protective immune response in generating antibodies to the S1S2 spike protein. Such a protocol may be used for other antigenic proteins in generating a liposomal vaccine. Adsorption may be achieved, without limitation through ionic, hydrophobic, Van der Waals interactions, hydrogen bonding, and/or through covalent interactions and/or conjugation. Methods of manufacture as described in further detail below may entrap the target antigen inside a liposome as well as decorating the liposome surface with spike proteins by adsorption through molecular interactions. Where at least a nanoparticle 104 includes a liposome, liposome composition may be chemically modified to an appropriate surface charge that maximizes binding of target antigen to surface of the liposome and for presentation of the liposomes to the immune cells.

In an embodiment, and still referring to FIG. 1B, antigen may include a combination of above-described elements. For instance, and without limitation, antigen may include a nucleic acid and an antigenic protein; nucleic acid may encode antigenic protein and/or may encode a different protein. In an embodiment, at least a nano particle may be combined with nucleic acid and antigenic protein in distinct ways and/or in distinct manufacturing steps. For example, and without limitation, a first antigen element, which may be either nucleic acid or protein, may be combined first with at least a nanoparticle using first combination step as described in further detail below, such as reconstitution of lyophilized nanoparticle with the first element, which may lead to entrapment of first element within nanoparticle, and second with a second element, which may be any of nucleic acid and protein, for instance by addition of second element after reconstitution of nanoparticle solution; a result may be entrapment of first element within nanoparticle while second element may be complexed with, attached on, embedded in, and/or complexed with lipid surface.

Referring now to FIG. 4, immunogenic composition may be manufactured, stored, and/or prepared in one or more lyophilized forms and/or in one or more dried states using various drying technologies such as without limitation spray drying, vacuum drying, foam drying, or the like. For instance, and without limitation, immunogenic composition and/or one or more components thereof may be presented in an on-demand format in which composition is lyophilized for stability, then reconstituted for use. For instance, and without limitation, immunogenic composition may be formulated as a lyophilized composition, after incorporation of antigen in at least a nanoparticle 104. Alternatively or additionally, nanoparticle delivery system may be lyophilized separately and reconstituted with the antigen; in other words, incorporation may be performed concurrently with reconstitution. Reconstitution may refer to resuspension, hydration, solvation, or otherwise reconstituted in aqueous solution, including buffer compositions such as phosphate-buffered saline (PBS), or the like. In further non-limiting illustrative embodiments, reconstitution of a lyophilized nanoparticle, such as a liposome-glycoprotein complex, may be performed with varying salt concentrations, such as sodium chloride. In an embodiment, varying salt concentrations may be encapsulated in any lipid layer of nanoparticle 104 as described throughout this disclosure, for example, a dried lipid core wherein reconstitution of the liposome may occur with the addition of an mRNA solution. In an embodiment, reconstitution of separately lyophilized nanoparticles with antigen may cause antigen to be trapped within a vesicle and/or other interior such as an aqueous interior of a liposome as well as attached to a surface thereof.

Still referring to FIG. 4, immunogenic composition 100 may include at least one lyoprotectant. A lyoprotectant, as used in this disclosure, is a substance that protects a substance during cryogenic freezing, during freeze-drying, and/or during freeze-thaw cycles. At least one lyoprotectant may include, without limitation, a polyol, such as without limitation sucrose, trehalose, mannitol, or the like, and/or at least one ionic strength balancing component, including for instance a salt, pH buffer, or the like. At least one lyoprotectant may include an amino acid, such as without limitation glycine, arginine, or the like. Persons skilled in the art, upon reviewing the entirety of this disclosure, will be aware of various alternative or additional lyoprotectants, cryoprotectants, and the like that may be employed consistently with this disclosure.

Still referring to FIG. 4, immunogenic composition 100 may include any suitable combination of elements including without limitation any set of formulations as set forth below in table 1. Formulations may include without limitation protectants such as sugar, pH control buffers, preservatives such as polysorbate 20%, and/or an ingredient such as NaCl or other salts to balance ionic strength. Polysorbate may generally be any concentration. Alternatively, in some embodiments, Poloxamers may be used instead of polysorbate.

TABLE 1
Exemplary Formulations

| | | | | | | Polysorbate | |
|---|---|---|---|---|---|---|---|
| Vaccine | S1 | S1-S2 | Lipid^{a} | pH | Buffer | 20% | Sugar |
| B-S1 | 10 μg/mL | — | 25 mg/mL | 7.2 | Histidine | 0.05 | 10% Sucrose |
| B-S1S2 | — | 10 μg/mL | 25 mg/mL | 7.2 | Histidine | 0.05 | 10% Sucrose |

^{an}including cholesterol 112 and alkyl amine.

Still referring to FIG. 4, vaccine may be administered in any suitable manner. In an embodiment, vaccine may be injection. Vaccine may alternatively or additionally be absorbed through a mucous membrane, for instance via aerosolized delivery to the nostrils and/or lungs. Alternatively or additionally, vaccine may be administered using a patch, such as without limitation a microneedle patch that delivers lyophilized vaccine in powder form; as a non-limiting example, lyophilized vaccine may be included in soluble microneedles which upon insertion to tissue of a living organism may dissolve in fluids thereof, reconstituting and activating the vaccine. As a further non-limiting example, lyophilized vaccine may be delivered in an implant such as a soluble or insoluble needle inserted under the skin or into other tissue allowing fluids of the subject tissue to reconstitute and disseminate the vaccine. Vaccine may be delivered in liquid and/or lyophilized form to any mucous membrane; for instance and without limitation, vaccine may be delivered as a lyophilized inhalable powder for absorption in nasal and/or pulmonary surfaces. Vaccine may be delivered orally, for instance in a needle or other device for injecting lyophilized vaccine into and/or across digestive tissues, which may be delivered in a capsule designed to disintegrate in one or more digestive juices. Vaccine in lyophilized form may be delivered by a nanobot.

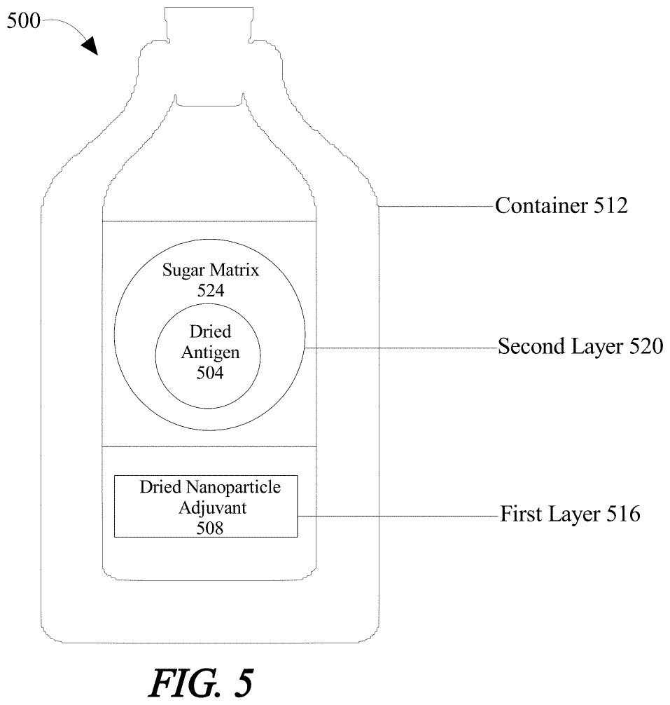

Referring now to FIG. 5, an exemplary diagram of combining a dried antigen 504 and a dried nanoparticle adjuvant 508 is provided. Combination may occur in any suitable container, including without limitation, beakers, flasks, test tubes, spot plates, crucibles, and the like. Dried antigen 504 and dried adjuvant may be deposited in the container as illustrated in FIG. 5. In some embodiments, two layers may be deposited into container 512. First layer 516 may be deposited containing a first selection of only one of dried antigen 504 and dried nanoparticle adjuvant 508. Second layer 520 may be deposited into container 512 containing a second selection of only one of dried antigen 504 and dried nanoparticle adjuvant 508 on top of first layer 516, wherein the first selection is distinct from the second selection. In some embodiments, the two layers may be separated by an impermeable film as described further below. In some embodiments, the first selection may contain dried nanoparticle adjuvant 508 and the second selection may contain dried antigen 504, such that a result is a layer of dried antigen 504 sitting on top of a layer of dried nanoparticle adjuvant 508; alternatively or additionally, the first selection may contain dried antigen 504 and the second selection may contain dried nanoparticle adjuvant 508, resulting in a layer of dried nanoparticle adjuvant 508 sitting on top of a layer of dried antigen 504. One or more layers and/or one or more of dried antigen 504 and dried nanoparticle adjuvant 508 may be embedded in additional material such as a sugar matrix. For instance, in some embodiments, depositing a top layer may include embedding dried antigen 504 in sugar matrix 524; dried antigen 504 may be isolated in this way to protect stability of its form during storage. Dried antigen 504 may be embedded through matrix isolation. “Matrix isolation” as used in this disclosure, is an experimental technique generally involving a material (e.g., dried antigen 504) being trapped within an unreactive matrix. A “host matrix,” as used in this disclosure, is a continuous solid and or amorphous phase in which guest particles (atoms, molecules, ions, etc.) are embedded. The guest is said to be isolated within the host matrix. Sugar matrix 524 may be composed from a plurality of monosaccharides, disaccharides, and polysaccharides acting as a host matrix. For example, and without limitation, matrix may be composed of glucose (dextrose), fructose, galactose, xylose, ribose, mannitol, sucrose, trehalose, mannose, lactose, any combination thereof, or the like. Additionally and alternatively, dried antigen 504 and nanoparticle adjuvant 508 may each be separately embedded in sugars, as described above, of different solubility to aid in controlled reconstitution and entrapment rates if there is no impermeable film to separate the two layers. In some embodiments, nanoparticle adjuvant 508 may be in the form of lyophilized beads for easy transfer into the syringe. Furthermore, lyophilized beads may offer an optimized ratio of volume to surface areas, resulting in faster reconstitution. In some embodiments, a diluent may be added to the top layer to dissolve the dried antigen first, thus creating an antigen solution. The antigen solution may then in sequence reconstitute the dried nanoparticle adjuvant to form a vaccine or drug delivery system. In some embodiments, a diluent may be added to the dried combination for reconstitution purposes. The diluent may be added to second layer 520 which would dissolve sugar matrix 524 and hydrate dried antigen 504 and form an antigen solution. Antigen solution may then reconstitute dried nanoparticle adjuvant 508 to form a vaccine or drug delivery system. Diluent may include any solution described throughout this disclosure alone or in combination. For example, phosphate-buffered saline (PBS), varying salt concentrations of sodium chloride, water for injection (WFI), sterile water, calcium carbonate, xanthan, and the like.

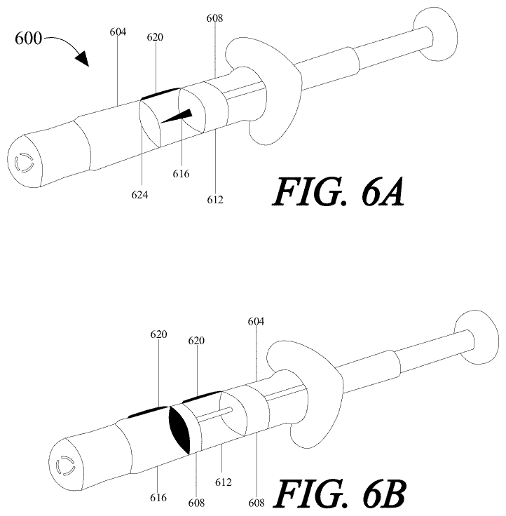

Referring now to FIGS. 6A and 6B, exemplary embodiments of an injection device to administer a vaccine are shown. In an embodiment, the vaccine formation may occur in a multi-chambered injection device such as dual-chamber and tri-chamber syringes. In multi-chamber syringes, lyophilization may occur prior to the placement of the antigen and nanoparticle 104 in the syringe or may occur while in the syringe by using any lyophilization process described throughout this disclosure.

In an embodiment where lyophilization occurs in the syringe, nanoparticle 104 may be deposited into a first chamber, forming the first layer. Nanoparticle 104 may be suspended in an aqueous medium, as described further below, thereafter a barrier may be placed on top of the first layer to separate the second layer to be deposited containing the antigen. The antigen may be suspended in an aqueous medium such as a buffer solution. The buffer may include any buffer solution described throughout this disclosure, for example and with without limitation, a lyoprotectant. The barrier may be sugar film 624 as discussed further below. Additionally, the barrier may be replaced by a breakaway stopper that can be pushed inward as a plunger rod moves down for reconstitution purposes. After the layers are added into the first chamber, the contents may then be dried by lyophilization or any-drying process.

In an embodiment where lyophilization occurs outside the syringe, the layers may be inserted into the syringe as a powder or beaded form. Nanoparticle 104 and the antigen may be lyophilized externally in a container, such as any container described in FIG. 5. The container may be shaped similar to a dual-chamber or tri-chamber syringe (e.g., a multi-chambered cartridge or a dual-chamber cartridge including the syringe embodiment of FIG. 6A, discussed further below) so that the dried contents of the container can be easily inserted into to the barrel shaft of a syringe. For example, the container may contain the layered antigen and nanoparticle 104 as discussed above and then be lyophilized. Lyophilization may include embedding the antigen and nanoparticle 104 in sugar matrixes or sugars of different solubility for thermal stability. A second chamber to the container may be filled with a diluent prior to the container being loaded into the syringe barrel. The container may then be aligned against the opening of the syringe barrel and pushed inside, wherein the plunger rod is inserted and thus locking the container into place. Alternatively, lyophilization occurring outside the syringe may include the antigen and nanoparticle 104 being lyophilized into packaging units by a container that molds the drug contents into stackable formats. This method may allow for numerous layers and layering combinations within the syringe. After lyophilization, each unit may be wrapped in a membrane made from at least sugars as described throughout this disclosure. The membrane may wrap around a unit in its entirety or cup the bottom and side of the unit. The wrapped units may then be inserted into the barrel shaft of a multi-chamber syringe. For example, in a dual chamber syringe, the first chamber may be layered with the stackable wrapped units.

Referring now to FIG. 6A, A dual-chamber syringe may have two partitioned chambers, where a first or front chamber 604 may be filled with a first product, such as without limitation a lyophilized drug product, which may include without limitation any lyophilized substance or combination of such substances and/or layers thereof as described in this disclosure, and a second or back chamber 608 with a second substance, which may include a diluent such as any diluent, suspension, and/or solution as described in this disclosure, or both chambers may be filled with drug products intended for co-delivery, among other possibilities. A syringe may contain a plunger and/or plunger rod at an end of a dual-chamber barrel, and a hub at an oppositive end of first chamber 604 that is connected to a disposable needle 616, which needle may be used for injection. In an embodiment, two or more layers of material, such as solid and/or freeze-dried material may be deposited into first chamber 604 of the syringe. First chamber 604 may include both nanoparticle 104 and the antigen, separated from each other by an impermeable film such as sugar film 624 containing a mixture of pullulan and trehalose. Trehalose is a disaccharide that is commonly used as a cryoprotectant and stabilizing agent. Pullulan is a water-soluble polysaccharide, consisting of maltotriose units and is produced from starch by the fungus Aureobasidium pullulans. Pullulan can be chemically converted to create a polymer that is either partially soluble or fully insoluble in water. Characteristic features of this polysaccharide may be due to its unique glycosidic linking. Pullulan may be easily altered chemically to decrease water solubility or to produce pH sensitivity, by presenting functional reactive groups or the like.

In some cases, there may be no sugar film separating the layers wherein nanoparticle 104 and the antigen may be lyophilized and embedded in sugars of different solubilities of one another as described in FIG. 5. Sugar film 624 may additionally be composed from a plurality of monosaccharides, disaccharides, and polysaccharides as described throughout this disclosure. The first layer may be deposited into first chamber 604 of the syringe containing a first selection of only one of the antigen and nanoparticle 104. The second layer may be deposited into first chamber 604 of the syringe containing a second selection of only one of the antigen and nanoparticle 104 on top of first layer, wherein the first selection is distinct from the second selection or separated by a film. For example, nanoparticle 104 may be selected for the first layer and the antigen may be in the second layer on top of the first.

In some cases, a second chamber 608 may be filled with a diluent, wherein second chamber 608 is separated from a first chamber 604 by at least a stopper, which may include a rubber stopper 612 with a window for liquid passage. The stopper may be made from elastic material containing elastomer such as natural rubbers, styrene-butadiene block copolymers, polyisoprene, polybutadiene, ethylene propylene rubber, ethylene propylene diene rubber, silicone elastomers, fluoroelastomers, polyurethane elastomers, nitrile rubbers, and any material suitable for use as a stopper as recognized by persons skilled in the art, and/or any material recognized as a suitable elastomeric material therefor. Rubber stopper 612 may contain a micro-needle 616 to punch into first chamber 604 and through sugar film 624 to allow the diluent to reconstitute the antigen. Micro-needle 616 may be multipronged, hollow, or include channels to allow easy movement of fluid. Reconstitution of the antigen may form an antigen solution that may then pass into the first layer through the punch hole in sugar film 624 and reconstitute nanoparticle 104. The diluent may include any solution described throughout this disclosure alone or in combination. For example, phosphate-buffered saline (PBS), varying salt concentrations of sodium chloride, water for injection (WFI), sterile water, calcium carbonate, xanthan, and the like. A user may push a plunger/plunger rod, made out of any material suitable for use as stopper, forward, forcing the diluent in second chamber 608 into first chamber 604 that may reconstitute the antigen to form an antigen solution. The antigen solution may then reconstitute nanoparticle 104 for vaccine delivery. The introduction of second chamber 608 content into first chamber 604 may build up pressure within the syringe. To counter this, the syringe may include at least external bypass 620 in first chamber 604. The bypass may be a channel that lets the diluent in second chamber 608 flow around rubber stopper 612 when the stopper is plunged into first chamber 604. As the diluent passes through the bypass, the antigen located in the second layer of first chamber 604 may be reconstituted to form the antigen solution. Similarly, in some embodiments, the bypass may be internal to the syringe such as a check valve located in rubber stopper 612. Check valves are two-port valves, meaning they have two openings in the body, one for fluid to enter and the other for fluid to leave. In some embodiments, to prevent the pressure from pushing the plunger/plunger rod back out, the plunger rod may have an intermediate lock that keeps the plunger rod in position after the contents of second chamber 608 are introduced into first chamber 604. Additionally, to prevent tampering with the plunger before administration of the vaccine, a screw down crown may be added around the of the plunger rod on top of a finger grip to the syringe. As used in this disclosure, a “screw down crown” is lock around the plunger rod that holds the rod in place when twisted in a particular direction. The screw down crown may be rigged for easy twisting and made from any material disclosed for the syringe, for example, stainless-steel.

Referring now to FIG. 6B, in some embodiments, a multi-chamber syringe may contain three chambers. The third chamber 604, which may include a top chamber closest to plunger, may contain the diluent wherein the third chamber 604 is connected to rubber stopper 608 containing a liquid passageway such a check valve, like the dual chamber syringe. In some embodiments, syringe may include a micro-needle as described in FIG. 6A. Second chamber 612 may contain the antigen separated from first chamber 616 containing a second rubber stopper 608 with a liquid passageway. In this embodiment, the tri-chamber syringe may contain at least external bypass 620 located on both second chamber 612 and first chamber 616. The antigen and nanoparticle 104 may be lyophilized prior or while in the syringe as described above. In this embodiment, the antigen and nanoparticles may be embedded in sugar matrices or films in their selected chambers for thermal stability during storage. For example, when a user applies pressure to the plunger, the diluent may be pushed through rubber stopper 608 into second chamber 612 by external bypass 620 to reconstitute the antigen to form an antigen solution. As the plunger is pushed further into first chamber 616, the antigen solution may enter a second external bypass 620 to reconstitute nanoparticle 104 and form the vaccine. Alternatively, second chamber 612 may contain nanoparticle 104 thereby being reconstituted first, forming a nanoparticle solution; solution may enter first chamber 616 containing dried antigen, using the same method as above, and reconstitute it to form a vaccine or any immunotherapy product described throughout this disclosure. Overall, an order in which reconstitution happens in a multi-chamber syringe may determine a location of antigen, such as the antigen being placed on a surface of a pre-made liposome or embedded in a pre-made liposome or neutral liposome. Additionally, this syringe may contain locking features like the dual chamber syringe. The locking feature may prevent the plunger from pushing back out and hold it in place as described previously. This embodiment may also include a screw down crown as described above.

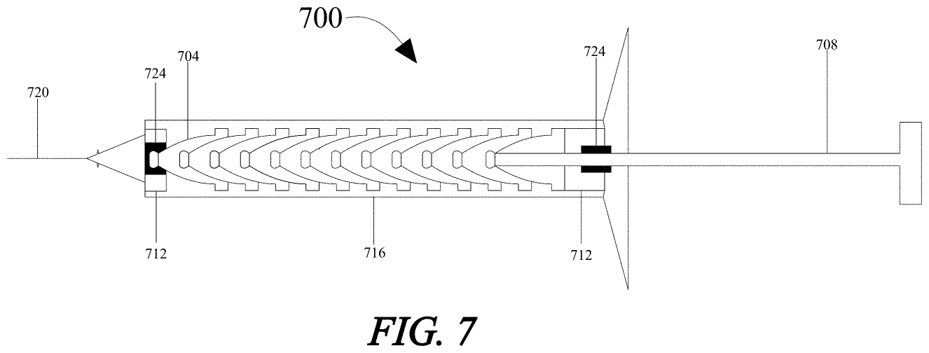

Referring now to FIG. 7, is an exemplary embodiment of an injection device to administer a vaccine. In an embodiment the vaccine delivery may occur in an injection device such as a disposable multi-packet syringe 700. The multi-packet syringe may house the vaccine within a plurality of single unit dosing packets 704, wherein each of the single unit dosing packets 704 may be separately disposed of after the vaccine housed therein is administered through the injection device. This pre-filled syringe may be designed to contain multiple packets 704 that may each be dispensed without breaking sterility. The packets 704 may be made of an array of synthetic and/or semi-synthetic material that use polymers as an ingredient (e.g., plastic), such as Acrylic, Polymethyl Methacrylate (PMMA), Polycarbonate (PC), Polyethylene (PE), Polypropylene (PP), Polyethylene Terephthalate (PETE or PET), Polyvinyl Chloride (PVC), and Acrylonitrile-Butadiene-Styrene (ABS). In some embodiments, the outer embodiment of multi-packet syringe 704 may be modeled after a traditional syringe, including plunger/plunger rod 708, stopper 712, hub, barrel shaft 716, and disposable needle 720. Plunger/plunger rod 708 and stopper 712 may be made from materials as described in FIG. 6A. Barrel shaft 716 may contain the multi-packets 704 wherein barrel shaft 716 may be made of plastics and rubbers as described as above, and additionally may be made of glass or stainless steel. Disposable needle 720 may be made of glass, polymer (plastic) or stainless steel.

This injection device may save material costs (drug product) for hospitals and clinics and make injections more efficient for surgeons. This could potentially save millions of dollars for healthcare providers, insurance companies and patients. In addition, the amount of medical waste may be decreased significantly. The pre-filled syringe may contain locking feature 724 that would enable each single unit cartridge or packet to be fully administered, while providing a means of disposing of each spent cartridge or packet while maintaining sterility of the device. The injection device may be designed for single unit doses that are multi-packed but contained within a central housing of the device. In some embodiments, several pre-filled units may be inserted in a pen and can be used on an individual, one-by-one basis. As each cartridge or packet is used, it may be discarded and the plunger may be pushed forward, which may allow the next cartridge or packet to be advanced in a ready-to-use position. A disposable needle may be connected for injection. The plunger may push forward as each individual cartridge or packet, or unit is used. Each cartridge or packet or unit may be sterile and used individually. In a multi-pack pen with individual cartridge or packet or unit doses may save on material, drug substance, packaging and required space during storage and transportation, and may also make it easier to store and use the product in hospital setting and in clinics, as compared to individually packed pre-filled syringes currently available. This device may make it possible to have a multi-dose product without jeopardizing the sterility. Such device may also be used in autoinjectors, pens and any injection device.

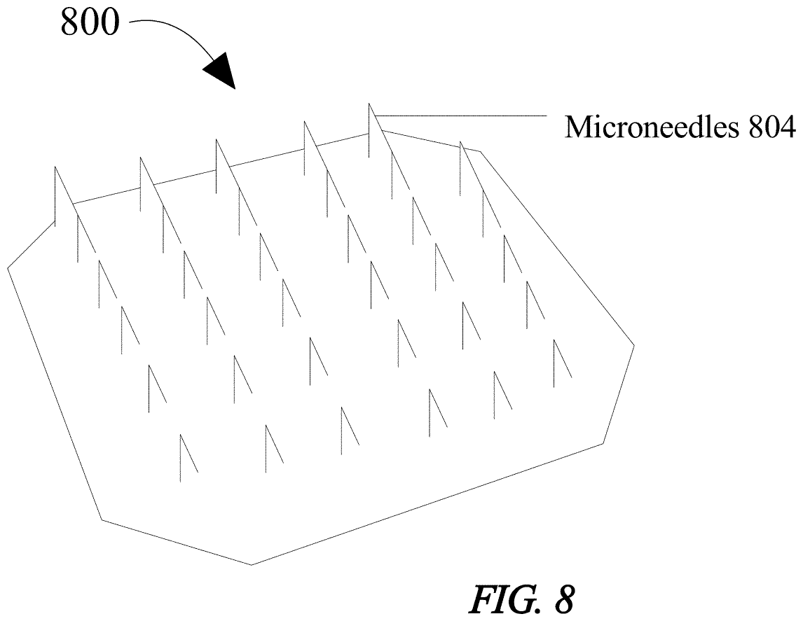

Referring now to FIG. 8, is an exemplary embodiment of an injection device to administer a vaccine is illustrated. In an embodiment the delivery may occur in an injection device such as a microneedle patch 800. Microneedle arrays are minimally invasive micron-sized needles that penetrate the stratum corneum, which is the skin's primary barrier to delivering a therapeutic through the skin. Microneedles 804 may vary between 50-900 microns in height and may be fabricated using various geometries and various metals, silicones, and polymers. The application of microneedle patches into the skin may form microscopic aqueous pores to allow the diffusion of drugs to the skin's epidermal layer. Microneedles 804 may be solid, dissolving, coated or hollow. Microneedles 804 may be used to deliver a liquid or lyophilized antigen, nanoparticle, and a nanoparticle adjuvant. Solid microneedles 804 may be used as a skin pretreatment. They may be inserted into the skin and then removed to form micron-sized pores on the skin surface. Drug solutions within a patch can then be applied to the surface, which contains the micropores. Hollow microneedles 804 are miniature versions of the conventional hypodermic needles. Drug delivery through hollow microneedles 804 may be achieved through a pressure-driven flow of a liquid formulation. Dissolving microneedles 804 may be made using biodegradable materials such as various polymers and sugars loaded with drug solutions. After the needle is applied to the skin, the needles may dissolve to release the payload (e.g., vaccine) into the skin. Coated microneedles 804 may consist of solid microneedles 804 that may be coated with a drug solution or dispersion. There may be various methods to produce coated microneedles 804, including dip coating, in which the microneedles 804 are “dipped” into the coating solution. Spray coating can also be used to coat the needles.

In a solid microneedle patch embodiment, microneedles 804 may be made of stainless-steel. These stainless-steel microneedles 804 may be dip-coated with various antigens, including antigen solutions as well as antigens encapsulated in nanoparticles, as described further below. The coated antigen may then get released into the skin layers upon administration of metal microneedles 804. In a hollow microneedle patch embodiment, microneedles 804 may contain the vaccine antigen, filled inside the hollow needles which upon administration, deliver the vaccine antigens into the skin. For example, microneedles 804 may contain the vaccine as described in this disclosure in a liquid form or a lyophilized form. The lyophilized vaccine may be reconstituted upon contact with tissue fluid or other diluent in vivo methods described throughout this disclosure. In some cases, the hollow microneedles 804 may be made of silica to facilitate the delivery of the antigen with or without an adjuvant encapsulated in nanoparticle 104. In some cases, the microneedles 804 may penetrate at a depth of at least 120 microns to deliver the vaccine an induce a humoral and cellular immune response.

In a dissolving microneedle patch embodiment, microneedles 804 may be composed of FDA approved polymers (e.g., polyvinyl alcohol (PVA), polyvinyl pyrrolidone (PVP), hyaluronic acid, polylactic acid) and can be loaded with the vaccine antigen or nanoparticles 104 containing the vaccine antigen. Upon administration, microneedles 804 may dissolve completely to release the vaccine into the skin. Dissolving microneedles 804 loaded with microparticles may have the advantage of the slow release of antigens as to achieve sustained release of antigen which may help in achieving a robust adaptive immune response. For example, a dissolving microneedle system made of polyvinyl pyrrolidone may deliver an encapsulated lyophilized antigen in nanoparticles 104. Dissolving microneedles 804 may also be used to maintain the antigen's stability at room temperature (25° C.) for more than one year. Additionally, microneedles 804 may be used to deliver combination vaccines in the form of a compartmental microneedle array (CMA). The CMA may be formulated with polylactic acid consisting of two separate sections in the same microneedle patch. For example, microneedles 804 formulated using CMC may be coated with spike proteins and their combination with liposomes, as described above, such as SARS-CoV-2, antigen, nanoparticle 104, etc., to produce significant levels of antigen-specific antibodies.

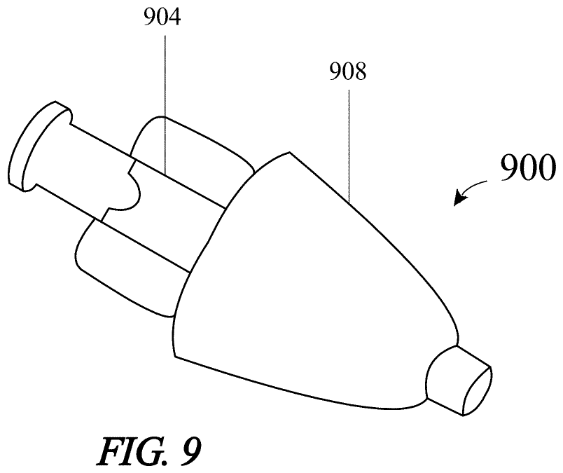

Referring now to FIG. 9, is an exemplary embodiment of a device attachment usable to administer a vaccine is illustrated. In some embodiments, an attachment may include a syringe spray adaptor 900 to deliver medication, such as a vaccine as described throughout this disclosure, for instance and without limitation as a nasal spray. Syringe spray adaptor 900 may be made from rubbers, metals, foams, and/or any other material described throughout this disclosure. Syringe spray adaptor 900 may be attached to a needle and/or microneedle end of a syringe as described throughout this disclosure, for example and with reference to FIGS. 6A, 6B, and 7. Syringe spray adaptor 900 may contain an atomizer and/or aerosolizer 904 in the center shaft to convert the vaccine into a fine mist. Syringe spray adaptor 900 may contain a nasal applicator 908. For example, with nasal applicator 908 placed against the entry of person's nasal passageway, when the plunger to syringe is fully pressed downward, the reconstituted vaccine may be converted into a fine mist to enter and coat the nasal mucous membrane. In some embodiments, syringe spray adaptor 900 may replace the needle/microneedle to a syringe and be screwed onto the end of the barrel shaft.

It should be noted that details of the foregoing injection device embodiments, given for purposes of illustration and are not to be construed as limiting the scope of this invention. Although several embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

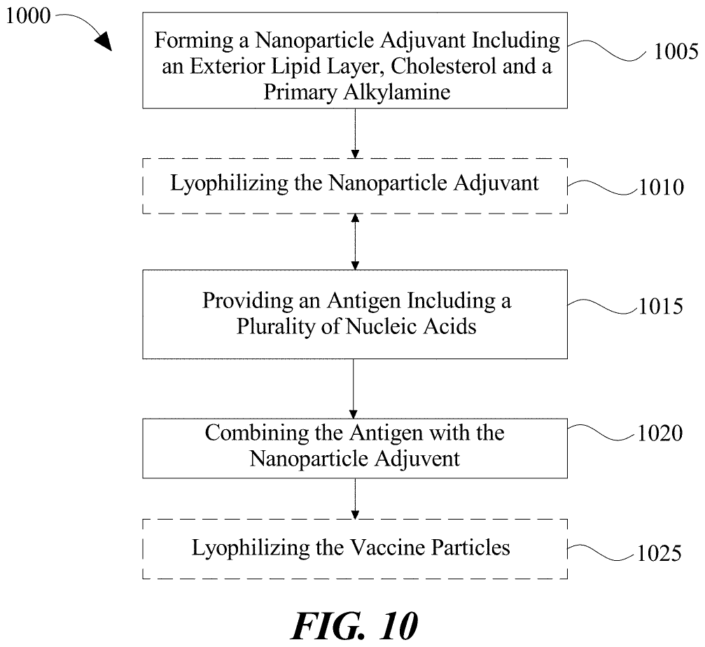

Referring now to FIG. 10, an exemplary embodiment of a method 1000 of manufacturing an immunogenic composition forming a vaccine is illustrated. At step 1005, a nanoparticle delivery system is formed. Nanoparticle delivery system may include any nanoparticle delivery system as described above. Nanoparticle delivery system includes a plurality of nanoparticles, which may include any nanoparticles as described above. Each nanoparticle includes a lipid layer 108 exterior including a plurality of lipids, cholesterol 112, and a primary alkyl amine 116 including a positively charged amino group head and at least a carbon tail, for instance and without limitation as described above. Lipid layer 108 may be positively charged, for instance by application of a concentration of a positively charged alkyl amine as described above. Each nanoparticle may include, without limitation, a liposome.

Still referring to FIG. 10, formation of nanoparticle delivery system may include formation of a suspension of liposomes. Formation may include hydrating a dried lipid blend, such as without limitation a freeze-dried lipid blend, and extruding the resulting solution through a filter having pore sizes at approximately an upper limit of a desired liposome diameter, which may be a desired diameter falling into ranges and/or average sizes as described above.

At optional step 1010, combining may include lyophilizing the nanoparticle delivery system, for instance and without limitation as described in further detail below.

At step 1015, and still referring to FIG. 10, method 1000 includes providing an antigen. Antigen may include a plurality of nucleic acids encoding a plurality of peptides from a coronavirus, as described above. For instance, and without limitation, nucleic acid may include a sequence encoding an S1 protein. Nucleic acid may include a sequence encoding an S1S2 protein. Antigen added with nanoparticle may include nucleic acid alone and/or in combination with spike protein. Intact antigen and/or various specific domains, such as S1 and S2 subunits may be recombinant; for instance, and without limitation, intact antigen and/or specific domains and/or subunits may be manufactured using mammalian cell-culture based expression systems, or a plurality of expression systems as described above. Alternatively or additionally, whole and/or partial virus particles may be generated and/or replicated, and spike proteins and/or subunits may be extracted, separated from, sheared off, or otherwise purified from such whole or partial virus particles, or virus-like particles.

With continued reference to FIG. 10, antigen may encode for a spike protein including a glycoprotein. Glycosylation of spike protein may occur during production and/or replication of virus particles. Glycosylation of spike protein may include providing glycosylated spike protein in immunogenic composition and/or spike protein forms encoded in nucleic acid with specific glycosylation sites. Glycosylation may be varied across batches and/or populations of spike proteins, among individual spike proteins, and/or among the different cell types that translate nucleic acid; this may generate a recombinant glycoprotein library with glycoproteins of varying degrees of glycosylation. In an embodiment, spike proteins of varying glycosylation may be combined in the antigen; this may result in nanoparticles, such as liposomes, incorporating a plurality of different glycoproteins of the same species. In an embodiment, such liposomes may allow for greater immunogenicity. For instance, and without limitation, preparation of S1 may include reconstituting S1 in water for injection (WFI) or formulation buffer to generate a given concentration, including without limitation a 250 μg/mL S1 stock solution. Specific amounts of S1 stock solution may then be diluted in specific amounts of a formulation buffer, which may include without limitation a 0.01% polysorbate 20/sucrose/histidine buffer to a concentration of approximately 10 μg/mL S1. As a further non-limiting example, a 550 μg/mL S1S2 stock solution, which may be reconstituted without limitation as described above, may be diluted in specific amount of formulation buffer, including any buffer as described above, to a concentration of approximately 10μg/mL S1S2. Buffer may generally include any buffer offering a buffering capacity within a pH range of 6.0-7.5, including without limitation histidine and/or phosphate buffer. Buffer may include a polysorbate 20 or 80 concentration within a range of 0.001%-0.05%. Buffer may include a polysorbate 20 or 80 concentration within a range of 0.001%-0.05%.

At step 1020, and still referring to FIG. 10, antigen is combined with nanoparticle delivery system. In an embodiment, a suspension of protein antigen may be added to an aqueous suspension of nanoparticle delivery system, using a mixing device to get a homogeneously distributed antigen-liposome mixture. Mixing device may include, without limitation, a magnetic stirrer, a sonication device, a homogenizer, or the like. Mixture may alternatively or additionally be swirled mechanically or manually. Combination according to this technique may tend to produce surface-mounted antigens and/or antigens adsorbed to lipid surface, for instance as described above. Combination according to this technique may tend to produce surface-attached antigen, antigen traversing lipid surface, and/or antigen encapsulated in lipid and/or aqueous core of lipid, for instance as described herein. Where nanoparticles have a charge with an opposite polarity to a charge of antigen, antigen may adsorb to the liposomes rapidly. In an embodiment, lyophilized nanoparticle delivery system may be reconstituted using a suspension of antigen in a buffer solution.

Alternatively or additionally, and continuing to refer to FIG. 10, hydration of lipid blend prior to extrusion may be performed with a solution and/or suspension of antigens; in other words, generation of nanoparticle delivery system may be performed concurrently with and/or subsequently to combination of antigen with nanoparticle delivery system. This may produce liposomes that include both entrapped and adsorbed antigens.

At step 1025, and still referring to FIG. 10, vaccine particles formed according to any process as described above may be lyophilized. Lyophilized vaccine particles may be delivered and/or stored in lyophilized form and may subsequently be reconstituted prior to administration and/or may be administered in powdered and/or lyophilized form as described above.

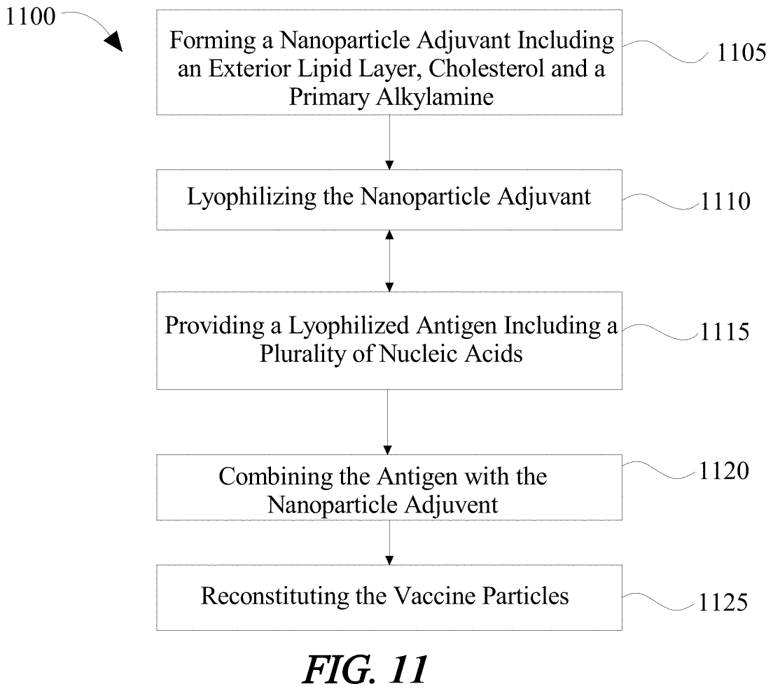

Referring now to FIG. 11, an exemplary embodiment of a method 1100 of manufacturing an immunogenic composition forming a vaccine is illustrated. At step 1105, a nanoparticle delivery system is formed. Nanoparticle delivery system may include any nanoparticle delivery system as described above. Nanoparticle delivery system includes a plurality of nanoparticles, which may include any nanoparticles as described above. Each nanoparticle includes a lipid layer 108 exterior including a plurality of lipids, cholesterol 112, and a primary alkyl amine 116 including a positively charged amino group head and at least a carbon tail, for instance and without limitation as described above. Lipid layer 108 may be positively charged, for instance by application of a concentration of a positively charged alkyl amine as described above. Each nanoparticle may include, without limitation, a liposome.

Still referring to FIG. 11, formation of nanoparticle delivery system may include formation of a suspension of liposomes. Formation may include hydrating a dried lipid blend, such as without limitation a freeze-dried lipid blend, and extruding the resulting solution through a filter having pore sizes at approximately an upper limit of a desired liposome diameter, which may be a desired diameter falling into ranges and/or average sizes as described above.

At step 1110, combining includes providing a dried nanoparticle delivery system which may include lyophilizing, any freeze-drying process, and/or any other drying process that would occur to a person skilled in the art upon reading the entirety of this disclosure, the nanoparticle delivery system.

At step 1115, and still referring to FIG. 11, method 1100 includes providing a dried antigen. Providing the dried antigen may include lyophilization, any freeze-drying process, and/or any other drying process that would occur to a person skilled in the art upon reading the entirety of this disclosure. Antigen may include a plurality of nucleic acids encoding a plurality of peptides from a coronavirus, as described above. For instance, and without limitation, nucleic acid may include a sequence encoding an S1 protein. Nucleic acid may include a sequence encoding an S1S2 protein. Antigen added with nanoparticle may include nucleic acid alone and/or in combination with spike protein. Intact antigen and/or various specific domains, such as S1 and S2 subunits may be recombinant; for instance, and without limitation, intact antigen and/or specific domains and/or subunits may be manufactured using mammalian cell-culture based expression systems, or a plurality of expression systems as described above. Alternatively or additionally, whole and/or partial virus particles may be generated and/or replicated, and spike proteins and/or subunits may be extracted, separated from, sheared off, or otherwise purified from such whole or partial virus particles, or virus-like particles.

With continued reference to FIG. 11, antigen may encode for a spike protein including a glycoprotein. Glycosylation of spike protein may occur during production and/or replication of virus particles. Glycosylation of spike protein may include providing glycosylated spike protein in immunogenic composition and/or spike protein forms encoded in nucleic acid with specific glycosylation sites. Glycosylation may be varied across batches and/or populations of spike proteins, among individual spike proteins, and/or among the different cell types that translate nucleic acid; this may generate a recombinant glycoprotein library with glycoproteins of varying degrees of glycosylation. In an embodiment, spike proteins of varying glycosylation may be combined in the antigen; this may result in nanoparticles, such as liposomes, incorporating a plurality of different glycoproteins of the same species. In an embodiment, such liposomes may allow for greater immunogenicity. For instance, and without limitation, preparation of S1 may include reconstituting S1 in water for injection (WFI) to generate a given concentration, including without limitation a 250 μg/mL S1 stock solution. Specific amounts of S1 stock solution may then be diluted in specific amounts of a formulation buffer, which may include without limitation a 0.01% polysorbate 20/sucrose/histidine buffer to a concentration of approximately 10 μg/mL S1. As a further non-limiting example, a 550 μg/mL S1S2 stock solution, which may be reconstituted without limitation as described above, may be diluted in specific amount of formulation buffer, including any buffer as described above, to a concentration of approximately 10 μg/mL S1S2. Buffer may generally include any buffer offering a buffering capacity within a pH range of 6.0-7.5, including without limitation histidine and/or phosphate buffer. Buffer may include a polysorbate 20 or 80 concentration within a range of 0.001%-0.05%. Buffer may include a polysorbate 20 or 80 concentration within a range of 0.001%-0.05%.

At step 1120, and still referring to FIG. 11, a dried antigen is combined with a dried nanoparticle delivery system. Combining may be achieved using any process described throughout this disclosure, for example and with reference to FIG. 5. Alternatively, a suspension of protein antigen may be added to an aqueous suspension of nanoparticle delivery system, using a mixing device to get a homogeneously distributed antigen-liposome mixture. Mixing device may include, without limitation, a magnetic stirrer, a sonication device, a homogenizer, or the like. Mixture may alternatively or additionally be swirled mechanically or manually. Combination according to this technique may tend to produce surface-mounted antigens and/or antigens adsorbed to lipid surface, for instance as described above. Combination according to this technique may tend to produce surface-attached antigen, antigen traversing lipid surface, and/or antigen encapsulated in lipid and/or aqueous core of lipid, for instance as described herein. Where nanoparticles have a charge with an opposite polarity to a charge of antigen, antigen may adsorb to the liposomes rapidly. In an embodiment, lyophilized nanoparticle delivery system may be reconstituted using a suspension of antigen in a buffer solution.

Alternatively, or additionally, and continuing to refer to FIG. 11, hydration of lipid blend prior to extrusion may be performed with a solution and/or suspension of antigens; in other words, generation of nanoparticle delivery system may be performed concurrently with and/or subsequently to combination of antigen with nanoparticle delivery system. This may produce liposomes that include both entrapped and adsorbed antigens.

At step 1125, and still referring to FIG. 11, lyophilized vaccine particles formed according to any process as described above may be delivered and/or stored in lyophilized form and may subsequently be reconstituted prior to administration and/or may be administered in powdered and/or lyophilized form as described above.

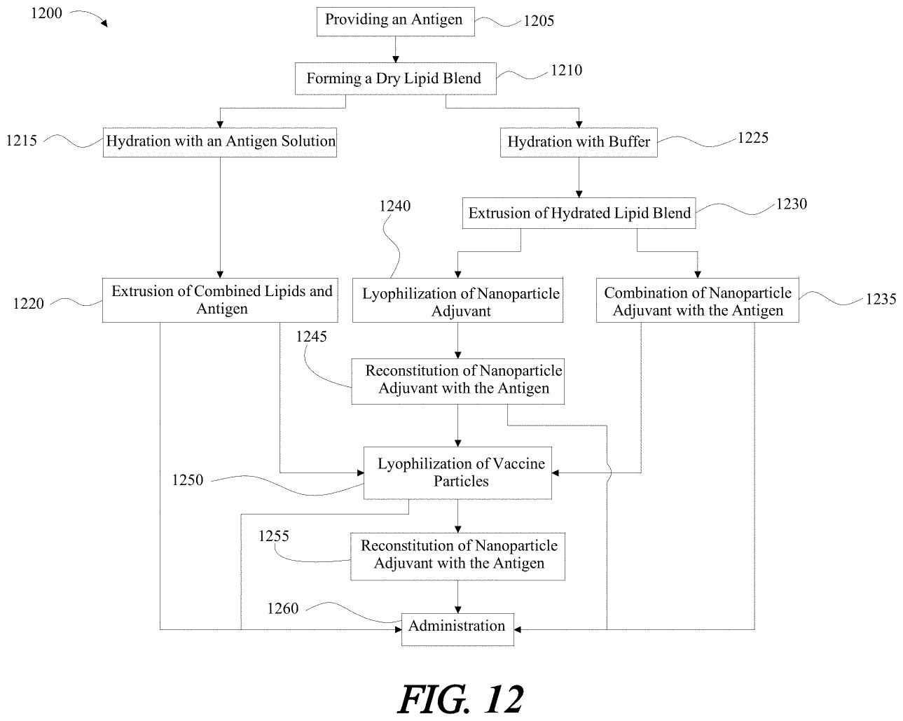

Referring now to FIG. 12, a flow diagram illustrating an exemplary embodiment of a method 1200 of manufacturing an immunogenic composition forming a vaccine is illustrated. At step 1205, an antigen is provided; antigen may include, without limitation, any antigen described above, including without limitation nucleic acid, spike proteins and/or glycoproteins of a coronavirus, and/or portions thereof. For instance, and without limitation, antigen may include nucleic acids encoding S1 glycoproteins and/or S1S2 glycoproteins. Provision of antigen may be performed, without limitation, according to any process described above in reference to FIGS. 1-11.

At step 1210, and still referring to FIG. 12, a dry lipid blend may be provided and/or formed. As a non-limiting example, a blend of DPPC, DOPC, cholesterol and stearylamine (40: 25: 20: 15:mol %, respectively may be dissolved in a chloroform/methanol/water solution. Solution may be dried, for instance in a rotary evaporator under a stream of nitrogen gas. Solution may subsequently be dissolved in a co-solvent of - cyclohexane/80% tertiary-Butyl alcohol (v/v) at a final lipid concentration of 20 mg/ml, for instance in aliquots of 50 mg of lipid/vial. Vials may then be lyophilized to obtain a dried lipid blend; vials may be sealed with nitrogen (N2) gas prior to partial placement of a stopper. Vials may then be freeze dried under a blanket of N2 gas, for instance in a freeze-dryer. As a non-limiting example lipid-blend may be lyophilized by freezing at −45° C., primary drying at −30 to −35C, and secondary drying at 25-30° C. Freeze-dried lipid blend may be powdered; this may increase surface area compared to film deposited on a vial according to conventional methods. It has further been found that lyophilization of lipid blend and/or nanoparticles has produced unexpectedly strong immune responses compared to conventional combinations that do not involve lyophilization in intermediate states of manufacture.

Further referring to FIG. 12, lipid blend may be hydrated with an antigen solution and/or suspension, as illustrated at step 1215. Antigen solution may include, without limitation antigen combined with a buffer to form a suspension. Buffer may include, without limitation, a lyoprotectant, which may include any lyoprotectant described above. As a non-limiting example, a 200 gr 10 mM histidine, 10% sucrose buffer may be prepared. A pH of buffer may be adjusted to approximately 7.2 when measured at 25 degrees Celsius. Buffer may be sterile filtered through a filter such as without limitation a 0.2 μm or 0.22 μm filter. Buffer may then be combined with the antigen mixture; alternatively or additionally, combination with antigen may occur concurrently with or subsequent to reconstitution of lyophilized nanoparticles with buffer. For instance, and without limitation, lyophilized lipid-blend may be hydrated with filtered antigen buffer solution and vortexed and/or sonicated until lipids are hydrated and liposomes are formed. Hydration with antigen solution may form a colloidal vaccine solution. At step 1220, colloidal vaccine solution may be extruded, for instance using filtration as described above in reference to FIG. 5, to form desired particle sizes. As a non-limiting example, where positively charged dried lipid-blends as described above, may be hydrated with a specific amount of a corresponding spike protein solution such as without limitation a 40 μg/mL spike protein solution; pH of spike protein solution may match pH of lipid and/or nanoparticle solution. A resulting combined solution may be extruded through filters; for instance, a vaccine particle solution may be extruded through a membrane filter, such as through 2×400 nm membrane filters in an extruder such as a 10 mL extruder. As a further non-limiting example, solution may be extruded ten times through two 400 nm polycarbonate filters in a 10 ml extruder at 50-100 psi using nitrogen gas. Extrusion may be performed gradually, for instance in a laminar flow hood using N2 gas. This procedure may be repeated one or more times; extrusion may be repeated until all solution has passed through the extruder 10 times. A resulting solution may be dispensed in vials; for instance, solution may be dispensed in 3 mL depydrogenated glass vials, for instance filling 800 μL fill volume. Dispensation may be performed in a laminar flow hood. Dispensation may be performed using a fine 1 mL pipette and sterile disposable pipette tips. Preparation according to steps 1215 and 1220 may be referred to herein as formulation “C”; for instance, where antigen is a solution of S1 spike proteins, formulation may be referred to as CS1, while where antigen is a solution of S1S2 spike proteins, formulation may be referred to as CS1S2.

Alternatively or additionally, and still referring to FIG. 12, at step 1225 dried lipid blend may be hydrated with formulation buffer, for instance and without limitation as described above, without antigen to form nanoparticle delivery system alone as a colloidal solution. For instance, and without limitation, lyophilized lipid-blend may be hydrated with filtered buffer, vortexed and/or sonicated until lipids are hydrated and liposomes are formed. Nanoparticle delivery system may be extruded and/or dispensed in vials as described above, as illustrated at step 1230.

In some embodiments, and with continued reference to FIG. 12, nanoparticle delivery system as formed at steps 1225 and 1230 may be combined in its form as a colloidal solution with antigen, for instance by mixing a protein solution of antigen with the colloidal solution of nanoparticles, as illustrated at step 1235; this may be implemented, without limitation, as described above in reference to FIG. 5. A formulation as described in reference to steps 1225, 1230, and 1235 is referred to herein as formulation “A”; for instance, where antigen is a solution of S1 spike proteins, formulation may be referred to as AS1, while where antigen is a solution of S1S2 spike proteins, formulation may be referred to as AS1S2.

Alternatively or additionally, and still referring to FIG. 12, nanoparticle delivery system may be lyophilized, as illustrated at step 1240. At step 1245, lyophilized nanoparticle delivery system may be reconstituted with antigen, for instance and without limitation using antigen in a buffered solution. A formulation as described in reference to steps 1225, 1240, and 1245 is referred to herein as formulation “B”; for instance, where antigen is a solution of S1 spike proteins, formulation may be referred to as BS1, while where antigen is a solution of S1S2 spike proteins, formulation may be referred to as BS1S2. In an embodiment, reconstitution of freeze-dried nanoparticles (“B”) with spike protein antigens to be trapped within a vesicle such as an aqueous interior of a liposome, as well as adsorbed to and/or trapped in lipid layer 108, for instance as illustrated above in reference to FIG. 4. In an embodiment, mixture of lyophilized delivery system with antigen may be performed shortly before administration; in other words, lyophilized delivery system and antigen may be transported and/or stored separately and combined at or near a site of administration.

At step 1250, and still referring to FIG. 12, any vaccine formulation described above may be lyophilized. Lyophilized vaccines may be denoted as formulation “D”; for instance, where antigen is a solution of S1 spike proteins, formulation may be referred to as DS1, while where antigen is a solution of S1S2 spike proteins, formulation may be referred to as DS1S2. At least one lyoprotectant as described above may be included with combination of antigen with nanoparticle delivery system. Lyophilization and/or inclusion of lyoprotectants may be accomplished in any manner consistent with descriptions provided above. For instance, and without limitation vaccines may be filled in vials and freeze-dried in a freeze-drier such as a Vertis Genesis 12XL by first freezing the solution to −45° C. at 0.5° C./min, followed by a 2-hour hold. Further continuing the example, primary drying may be performed below the primary glass transition of the frozen solution (Tg′), for example at −35° C. shelf temperature for at least 10 hours at a chamber pressure of 100 mTorr or until completion of primary drying. After primary drying, and still continuing the example, a shelf may be ramped up to 25° C. at 0.2° C./min. Still continuing the example, secondary drying may be performed at 25° C. shelf temperature for 4 hours at a chamber pressure of 100 mTorr. Second lyophilization of combined proteins and liposomes may cause a complex interaction between antigen, such as S1 or S1S2, and lipids and/or sugar and/or other lyoprotectant. At step 1255, freeze-dried vaccines, such as freeze-dried S1 and S1S2 liposomal vaccines (referred to here as D-S1 and D-S1S2) may be reconstituted with water for injection (WFI). A resulting liposome solution may include a 25 mg/mL liposome (or between 1 and 50 mg/mL) and 10 ug/mL S1 or S1S2. Alternatively, lyophilized vaccine may be directly administered.

At step 1260, vaccine may be administered, according to any suitable process for administration, including without limitation any process described in this disclosure. The above-described methods are provided for exemplary purposes only; any combination of method steps as described in this disclosure is considered within the scope of this disclosure.

Reference is now made to immunization study results regarding study of immunization to coronavirus in mice. Study was conducted according to an approved Animal Care and Use Protocol (ACUP). 2×50 μl of each formulation tested was injected intramuscularly (im) in the leg of five female BALB-C mice on days 0 and 14. Serum was collected from the immunized mice as well as naïve mice (negative control; non-vaccine injected) on Days 14 and 28. Five mice were tested per group.

An antibody response to each vaccine was determined using an Indirect Enzyme-Linked Immunosorbent Assay (ELISA) method that was designed for the detection of mouse antibodies against SARS-CoV-2 spike proteins. Each microtiter plate (Coster 3369, EIA/RIA Plate) was coated with 0.1 μg of S1 per well or 0.2 μg of S1/S2 per well; testing indicated that use of 0.1 μg of S1/S2 produced similar results. Sera from mice were diluted 100-fold in blocking buffer (0.5% Bovine albumin serum (BSA) in 0.05% Polysorbate 20-20). The diluted sera were serially diluted in duplicate to a final dilution of 6,400 times the initial sera. The plates were incubated at 5° C. overnight (16-18 hours). After washing the plates, horseradish peroxidase-conjugated Goat anti-mouse IgG secondary Antibody (HRP), Sino Biological) was diluted (1 μL/10 mL) in blocking buffer and 100 ul was added to each well to detect the antibodies against spike protein. After a 1-2-hour incubation at 37° C., the plates were washed and tetramethylbenzidine (TMB) substrate was added to detect Ab responses. The reaction was stopped after approximately 5 minutes with 1 N HCl. Immediately, absorbance was measured at 450 nm using a Spectromax 190 microplate reader (Molecular Devices, CA). Endpoint titer for each mouse was determined as the highest dilution of immune serum producing ELISA values (A450 nm) greater than or equal to five times the binding detected with a corresponding dilution of naïve mice sera. The mean A450 values obtained for the antibodies were calculated for each group of mice per vaccine. In all cases the results are the mean value of IgG titer absorbance for five mice. Samples were stored at 5 C, 25 C, and 40 C up to one month for stability evaluation. Stability was assessed by measurement of particle size and UV absorbance. Freeze-dried vaccine was found to be stable for at least two weeks at 40 degrees C., and one month at 25 degrees; as a result, vaccine may be suitable for transport and storage without refrigeration.

All samples were analyzed on a Precision Detector Dynamic Light Scattering (DLS) instrument PD2000DLS^{plus} and PDDLS/CoolBatch 90T using quartz cuvettes (Precision Detectors). Liposomal samples were diluted 197 times in histidine sucrose buffer, from an original 25 mg/ml suspension. Measurements were done at 20° C. using a refractive index of 1.3479 and a viscosity of 0.0133 Poise for a 10% sucrose solution. Sample time was 15 μsec with a 3 sec run duration and a total of 60 accumulations per measurement. Data was analyzed using Precision Deconvolve software. Stock solutions of S1 and S1S2 (at 250 and 550 μg/mL, respectively) and also 10 μg/mL solutions of S1 and S1S2 were also analyzed without dilution. Particle size was found to be stable between samples.

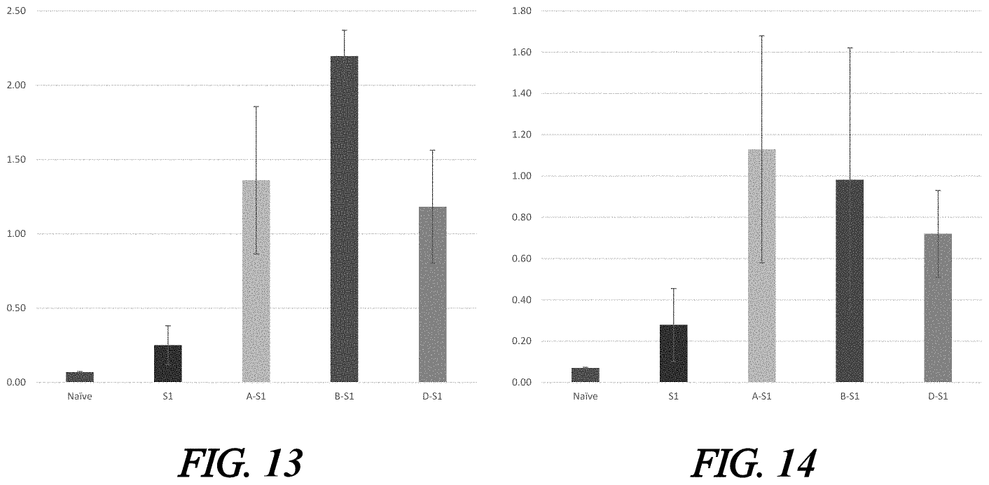

Referring now to FIG. 13, a bar graph illustrates experimental results comparing IgG immune response (vertical axis) measured from sera extracted from mice vaccinated using embodiments of disclosed vaccine in which the antigen was an S1 protein without S2 protein component. ELISA was performed with S1-immobilized plates. Naïve samples (unvaccinated) were compared to sera samples from mice vaccinated with four other formulations of a solution of S1 glycoproteins, denoted “S1,”, a vaccine formulated using a lipid blend that has not be lyophilized, which was mixed with antigens (in this case S1), which process is referred to in the graphs as “A-S1” as described above in reference to FIG. 12, a formulation of lipid blend lyophilized and reconstituted with S1 spike proteins, referred to in the graphs as “B-S1” as described above in reference to FIG. 12, and a formulation of freeze-dried lipid blend reconstituted with spike protein (S1) solution, then freeze-dried again, and reconstituted a second time, denoted formulation “D-S1” as described above in reference to FIG. 12. As shown in FIG. 12, A-S1, B-S1, and D-S1 all formulations significantly outperformed S1 alone and control resulting in a statistically significant increase in S1-neutralizing antibody response, while B-S1 outperformed A-S1 and D-S1 by a significant margin.

Referring now to FIG. 14, a bar graph illustrates experimental results comparing immune response (vertical axis) measured for sera from mice vaccinated using embodiments of disclosed vaccine in which the antigen was an S1 with no S2, placed on plates coated with S1S2. As before naïve control, S1, A-S1, B-S1, and D-S1 solutions were used. As illustrated in FIG. 10, all three vaccines were immunogenic.

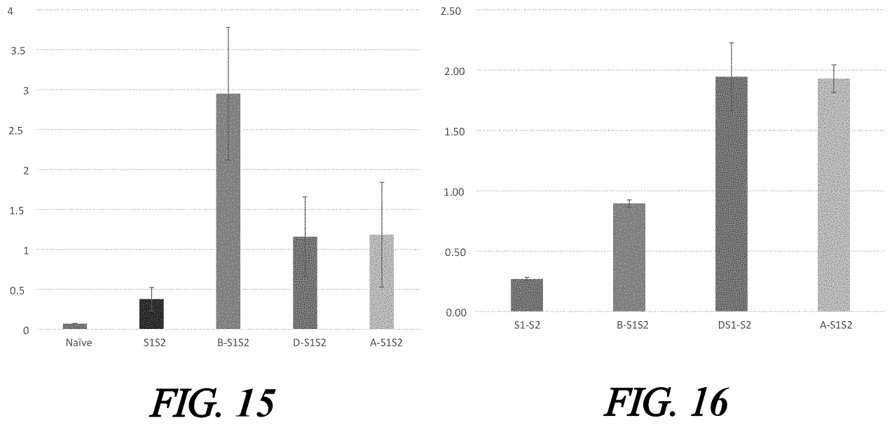

Referring now to FIG. 15, a bar graph illustrates experimental results comparing immune response (vertical axis) measured for blood from mice vaccinated using embodiments of disclosed vaccine in which the antigen was an S1S2 vaccine on a plate coated with S1. Formulations included “S1S2,” which was a solution of S1S2 alone, “A-S1S2,” prepared as described above for A-S1, but with S1S2 spike proteins instead of S1 alone, “B-S1S2,” prepared as described above for B-S1, but with S1S2 spike proteins instead of S1 alone, and “D-S1S2,” prepared as described above for D-S1, but with S1S2 spike proteins instead of S1 alone. All formulations were significantly more immunogenic than control, with B-S1S2 far outperforming others.

Referring now to FIG. 16, a bar graph illustrates experimental results comparing immune response (vertical axis) measured for blood from mice vaccinated using embodiments of disclosed vaccine in which the antigen was an S1S2 vaccine on a plate coated with S1S2. Formulations included S1S2, A-S1S2, B-S1S2, and D-S1S2. All formulations were significantly more immunogenic than control, with D-S1S2 and A-S1S2 outperforming B-S1S2.

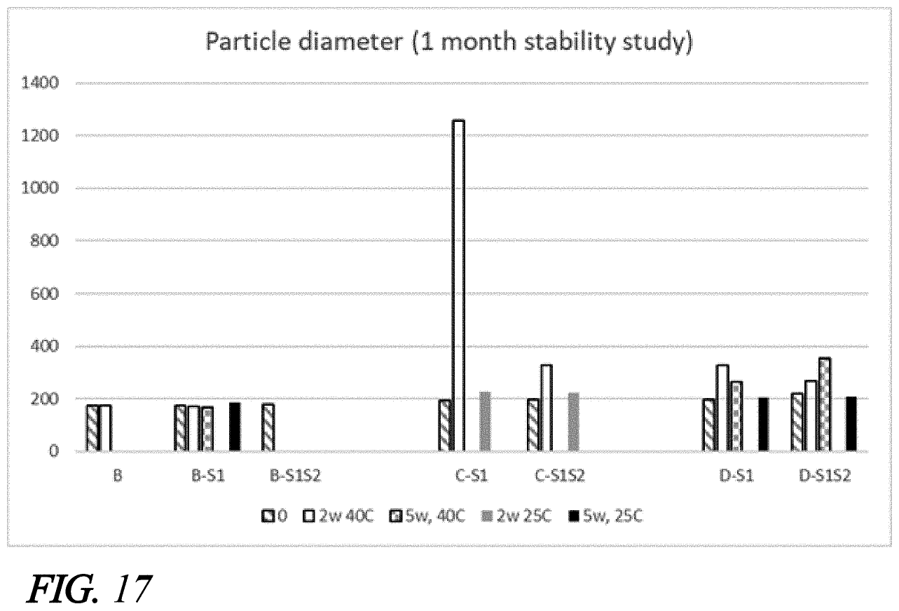

Referring now to FIG. 17, a graph showing experimental results of stability assessments at one month as described above is provided. Formulations C-S1 and C-S1S2 were not lyophilized. Formulations B-S1 and B-S1S2, which have been described above, were reconstituted, with antigens, as described above at a time of stability assessment. Formulations D-S1 and D-S1S2 were reconstituted at a time of assessment as well. As illustrated in FIG. 17 and shown in Table 1 with respect to particle size, lyophilized vaccines (D-S1 and D-S1S2) and lyophilized delivery system (B) were stable at 25 C for at least 5 weeks, and at 40C for at least 2 weeks. When lyophilized delivery system B was reconstituted at t0 with S1 protein, it resulted in a vaccine particle with a size that did not change significantly if the delivery system was stored for 5 weeks at 25 or for 2 weeks at 40 C. This would indicate that the liposomal delivery system B is stable at 25 for at least 5 weeks and at 40 C for at least 2 weeks.

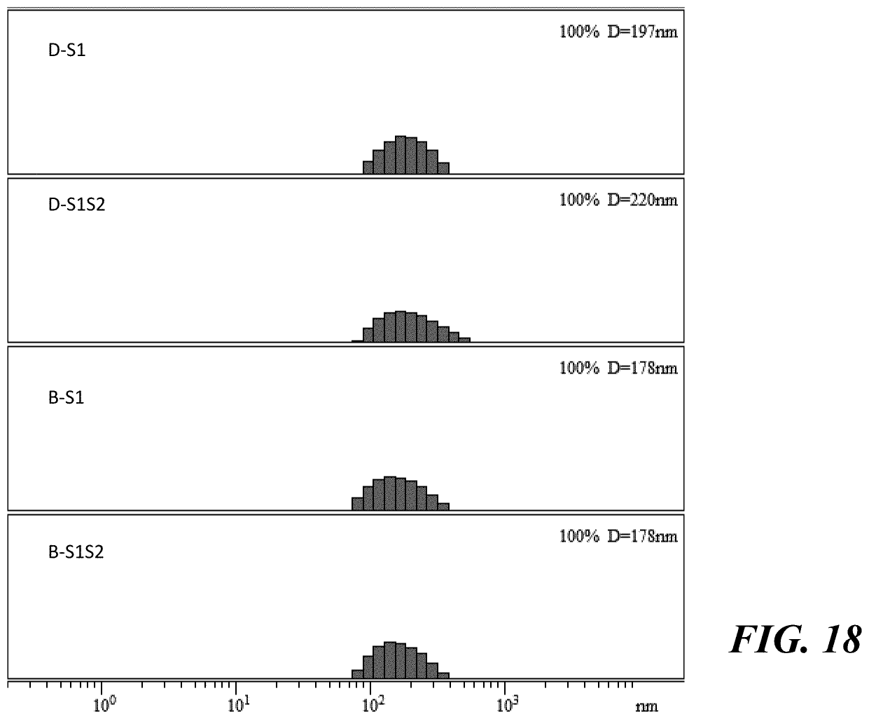

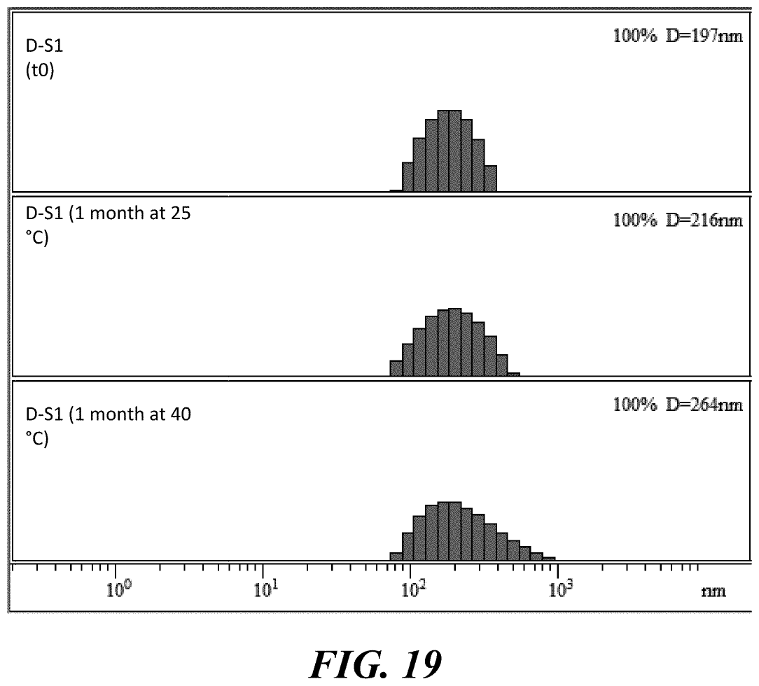

Referring now to FIGS. 18 and 19, representative histograms illustrating the effects of temperature on stability of B and D formulations, respectively. Lyophilization increased vaccine stability at 25° C. and 40° C. FIG. 18 illustrates by dynamic light scattering (DLS) experimentation that particle diameter is not significantly altered between vaccine formulation by the addition of different antigens. FIG. 19 illustrates by DLS that particle diameter may not be significantly altered by expended periods of time (1 month) at ambient room temperature (25° C.) or at elevated temperature (40° C.), demonstrating the stability of the nanoparticle formulation outside of the cold chain. Liquid vaccines C-S1 and C-S1S2 were not stable at either temperature for 2 weeks.

TABLE 1

| Vaccine | t0 | 2 w 25 C. | 5 w, 25 C. | 2 w 40 C. | 5 w, 40 C. |
|---|---|---|---|---|---|
| B | 176 ± 3 | | | 173 ± 6 | |
| B-S1 | 176 ± 3 | — | 186 ± 6 | 170 ± 2 | 166 ± 3 |
| B-S1S2 | 178 ± 2 | — | — | — | — |
| C-S1 | 193 ± 4 | 225 ± 5 | — | 1,257 ± 266 | — |
| C-S1S2 | 196 ± 3 | 221 ± 3 | — | 326 ± 30 | — |
| D-S1 | 198 ± 4 | — | 203 ± 16 | 326 ± 74 | 262 ± 9 |
| D-S1S2 | 218 ± 7 | | 209 ± 5 | 269 ± 22 | 355 ± 52 |

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve embodiments according to this disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A method of manufacturing an immunogenic composition forming a vaccine, the method comprising:
providing an antigen;
providing a dry lipid blend;
hydrating the dry lipid blend with an antigen solution, wherein the hydration is configured to form a colloidal vaccine solution; and
extruding the colloidal vaccine solution, wherein the extrusion is configured to form a vaccine particle.

2. The method of claim 1, wherein the dry lipid blend comprises a powdered dry lipid blend.

3. The method of claim 1, wherein the dry lipid blend comprises a positively charged dry lipid blend.

4. The method of claim 1, wherein the antigen solution comprises a nucleic acid, wherein the nucleic acid comprises at least a sequence of ribonucleic acid (RNA.)

5. The method of claim 5, wherein the at least a sequence of RNA further comprises at least a sequence of mRNA.

6. The method of claim 5, wherein the nucleic acid encodes at least a part of the antigen.

7. The method of claim 5, wherein the antigen comprises a spike protein, wherein the spike protein comprises an S1 protein.

8. The method of claim 1, further comprising:
lyophilizing the vaccine particle; and
reconstituting the lyophilized vaccine particle with the S1 protein.

9. The method of claim 8, further comprising:
lyophilizing the vaccine particle for a second time; and
reconstituting the vaccine particle with the antigen solution for the second time.

10. The method of claim 8, wherein the spike protein further comprises an S1S2 protein.

11. The method of claim 1, wherein the antigen solution comprises a combination of the spike protein and a buffer.

12. A method of manufacturing an immunogenic composition forming a vaccine, the method comprising:
providing an antigen;
providing a dry lipid blend;
forming a nanoparticle delivery system, further comprising;
hydrating the dry lipid blend with a buffer;
extruding the hydrated lipid blend; and
combining the nanoparticle delivery system with the antigen, wherein the combination is configured to form a vaccine particle;
lyophilizing the nanoparticle delivery system; and
reconstituting the lyophilized nanoparticle delivery system.

13. The method of claim 12, wherein the dry lipid blend comprises a powdered dry lipid blend.

14. The method of claim 12, wherein the dry lipid blend comprises a positively charged dry lipid blend.

15. The method of claim 12, wherein the antigen comprises a nucleic acid, wherein the nucleic acid comprises at least a sequence of ribonucleic acid (RNA.)

16. The method of claim 15, wherein the at least a sequence of RNA further comprises at least a sequence of mRNA.

17. The method of claim 15, wherein the nucleic acid encodes at least a part of the antigen.

18. The method of claim 17, wherein the antigen comprises a spike protein,

19. The method of claim 18, wherein the spike protein comprises an S1 protein.

20. The method of claim 18, wherein the spike protein further comprises an S1S2 protein.
